(19) United States
(12) Patent Application Publication
(10) Pub. No.: US 2008/0176254 A1
(43) Pub. Date: Jul. 24, 2008

(54) AFFINITY SEPARATION COMPOSITIONS AND METHODS

(76) Inventors: Xiangming Fang, San Diego, CA (US); Lei Huang, San Diego, CA (US); Ping Lin, San Diego, CA (US); Jerald S. Feitelson, San Diego, CA (US); Wei-Wei Zhang, San Diego, CA (US)

Correspondence Address: PERKINS COIE LLP, POST OFFICE BOX 1208, SEATTLE, WA, 98111-1208, US

(21) Appl. No.: 11/874,884

(22) Filed: Oct. 18, 2007

Related U.S. Application Data
(63) Continuation-in-part of application No. 10/892,423, filed on Jul. 14, 2004, now abandoned.
(60) Provisional application No. 60/487,528, filed on Jul. 14, 2003.

(51) Int. Cl. G01N 33/53; G01N 33/543; G01N 33/563; G01N 33/553; G01N 33/544; G01N 33/545

(52) U.S. Cl. 435/7.21; 436/518; 436/513; 436/526; 436/529; 436/531

(57) ABSTRACT

The present invention provides compositions and methods for affinity separation of targets from mixtures. In particular, disclosed are avian IgY antibodies coupled to solid supports and their methods of use.

### CROSS-REFERENCE TO RELATED APPLICATIONS

This application is a continuation-in-part of U.S. patent application Ser. No. 10/892,423, filed Jul. 14, 2004, which claims the benefit of U.S. Provisional Patent Application Ser. No. 60/487,528, filed Jul. 14, 2003, the disclosures of both of which are incorporated herein by reference, including drawings.

### FIELD OF THE INVENTION

The present invention relates to affinity separation of biological materials. The invention further relates to compositions of affinity reagents linked to solid supports and the methods that the solid support mediates affinity reagents to separate targets from non-targets in mixtures of biological samples. More specifically, the present invention relates to polyclonal avian IgY antibody compositions and methods of making and using them. The IgY antibodies are covalently bound in an oriented fashion to a solid support via carbohydrates in their Fc region, making the Fab regions of antibodies readily available for reaction with an antigen. The polyclonal IgY antibodies are useful for immunoaffinity capture, separation, purification and detection of a desired protein target in a complex mixture.

### BACKGROUND OF THE INVENTION

The dynamic range of protein concentration spreads from 7 to 8 orders of magnitude in cells and probably up to 12 orders of magnitude in plasma (Corthals, G. L., et al. Electrophoresis 2000, 21, 1104-1115; Anderson, N. L. & Anderson N. G., Mol Cell Proteomics 2002, 1, 845-867). Classical silver-stained two-dimensional electrophoresis gel (2DE) can only display up to four orders of magnitude (Adkins, J. N., et al, Mol Cell Proteomics 2002, 1, 947-955; Gygi, S. P., et al., Proc Natl Acad Sci USA 2000, 15, 9390-9395). This is a significant limitation for the discovery and analysis of important proteins such as regulatory proteins, cytokines, biomarkers, drug targets, etc. Plasma proteome research faces the challenge that the 10-15 most abundant proteins at the mg/ml level are only less than 0.1% of various types of proteins, yet they constitute more than 95% of the mass of total plasma proteins. The important proteins and biomarkers for malignant or non-malignant diseases (e.g. C-reactive protein, osteopotin, prostate-specific antigen, various interleukins and cytokines) are usually at ng/ml to pg/ml levels, making them like needles buried in a huge haystack of abundant proteins (Lopez, M. F., Electrophoresis 2000, 21, 1082-1093; Burtis, C. A. & Ashwood E. R., 2001. Tietz Fundamentals of Clinical Chemistry, 5^{th} Ed., W. B. Saunders Company, Philadelphia). Scientists, especially those who use 2DE and mass spectrometry (MS), require tools or reagents that can specifically separate the abundant proteins and prepare samples for attaining higher sensitivity and better resolution to detect low abundant proteins and thereby “dig deeper into the proteome” (Adkins, J. N., op cit). There is also an unmet need for reference standards for selectively removing high-abundant proteins from plasma to enrich the relatively very low-abundant proteins, thereby increasing their resolution of detection (Zhan, X. & Desiderio D. M., Proteomics 2003, 3, 699-713). Satisfying these demands will make it possible to more precisely measure the low-abundant proteins, particularly in multiplex settings for proteomic profiling (Lee, H., et al., Anal Chem 2002, 74, 4353-4360).

### Affinity Separation: Unmet Needs for Proteomic R&D

The Plasma Proteome Project (PPP) of the HUPO (Human Proteome Organization) recognized the challenge of the huge dynamic range of plasma protein concentration in conducting protein discovery research. Therefore, the PPP promoted development of methods and tools for protein fractionation and sample preparation at the inception of the project (HUPO Workshop and Planning Session, Apr. 29, 2002 Bethesda, Md., USA). To best meet the needs of proteomic analysis by 2DE and MS, sample preparation tools for pre-separation or pre-fractionation of proteins should have at least the following features:

1. High specificity and accuracy

2. Low cross-reactivity to other serum proteins

3. Strong avidity (high affinity)

4. High capacity

5. Multiple-species applicability

6. Convenient use

7. High reproducibility

8. Good reusability

9. Minimal disruption to natural condition of sample

10. Reasonable affordability

There are a number of approaches to separate proteins based upon their biochemical and biophysical features such as molecular weight, mass, density, hydrophobicity, surface charge, isoelectric point, tertiary structure, amino acid sequence (epitope), etc. Conventional centrifugation, ultrafiltration, and liquid chromatography, including textile dye ligands (Cibacron Blue), have been previously used to remove albumin and other plasma proteins (Travis, J., et al., Biochem J 1976, 157, 301-306; Rengarajan. K., et al., Biotechniques 1996, 20, 30-32; Butt, A., et al., Proteomics 2001, 1, 42-53; Georgiou, H. M., et al., Proteomics 2001, 1, 1503-1506; Tirumalai, R. S., et al., Mol. Cell. Proteomics 2003, 2, 1096-1103; Pieper, R., et al., Proteomics 2003, 3, 1345-1364; Lescuyer, P., et al., Electrophoresis 2004, 25, 1125-1135; Rothemund, D. L., et al., Proteomics 2003, 3, 279-87; Davidsson, P., et al., Rapid Commun Mass Spectrom 2002, 16, 2083-2088). However, these separation processes are not protein-specific and have variable capacity and limited reproducibility.

In comparison, affinity separation is a process specific to selected target proteins. Antibodies, proteins, peptides, nucleotides, etc., are affinity reagents that have been applied for this purpose. Bacterial Protein A and Protein G, which specifically bind to the Fc region of IgG, have been successfully used for specific separation from serum or plasma (Adkins, J. N op cit; Vesterberg, O. & Anundi H., Appl Theor Electrophor 1991, 2, 159-161). One of the limitations with protein-based affinity reagents, including affinity peptides generated via phage-display, is the limited diversity of products available for various target proteins. Antibody-based separation of proteins, known as immunoaffinity separation, recently became the method of choice due to its specificity and straightforward production. Immunoaffinity separation of proteins using different types of antibodies has generated encouraging data (Burgess-Cassler, A., et al., Clin Chim Acta 1989, 31, 359-365; Kojima, K., J Biochem Biophys Methods 2001, 49, 241-251; Pieper, R., et al., Proteomics 2003, 3, 422-432; Fang, X., et al., in: Reiner, J., et al. (Eds.), Frontiers of Biotechnology and Pharmaceuticals, Vol. 4, Science Press USA, Inc. Monmouth Junction, 2003, pp. 222-245). Commercial kits using either mammalian Immunoglobulin G (IgG) or avian Immunoglobulin Yolk (IgY) have also recently been made available for immunoaffinity depletion of albumin and some other abundant proteins, such as the kits marketed by Agilent, Amersham, Bio-Rad, GenWay Biotech, Pierce, Sigma-Aldrich, and others.

IgY antibody is immunoglobulin isolated from egg yolks (so called IgY) of the lower vertebrates, such as birds, reptiles, and amphibians (Leslie, G. A. & Clem L. W., J Exp Med 1969, 130, 1337-1352; Hadge, D. & Ambrosius H., Mol Immun 1984, 21, 699-707; Du Pasquier, L., et al., Annu Rev Immunol 1989, 7, 251-275). Avian IgY antibodies have been developed and successfully applied for various types of immunoassays (Larsson, A. & Mellerstedt H., Hybridoma 1992, 11, 33-39; Larsson, A., et al., Poultry Science 1993, 72, 1807-1812; Warr, G. W., et al., Immunol Today 1995, 16, 392-398; Schade, R. & Hlinak A., ALTEX 1996, 13, 5-9; Zhang, W.-W., Drug Discovery Today 2003, 8, 364-371). An outstanding advantage of avian IgY antibodies is that they are secreted by hens into egg yolk, resulting in a high-yielding reservoir of easy-to-access antibodies (Patterson, R., et al., J Immunol 1962, 89, 272-278). Compared to drawing blood, collecting eggs is non-invasive, continuous, convenient, and scalable. One egg yolk contains about 100 mg of total IgY. After a primary injection and three boosts, one hen can produce 40-60 eggs, yielding about 5 grams of antibodies. Distinct from IgG antibodies in molecular structure and biochemical features, IgY antibodies were shown to have several advantages over IgG, particularly for their high avidity and less cross-reactivity to human proteins (Stuart, C. A., et al., Anal Biochem 1988, 173, 142-150; Gassmann, M., et al., FASEB J 1990, 4, 2528-2532; Larsson, A., et al., Clin Chem 1991, 37, 411-414). Unlike IgG, the IgY Fc region does not bind human proteins such as complements, rheumatoid factor, Fc receptor, IgM, etc, significantly increasing IgY's specificity of capture.

### Non-IgY Affinity Separation Technologies

For introduction and general comparison, non-IgY affinity separation products for proteomic sample processing are reviewed based upon available published information.

### Albumin-Removal Blue-Dye Products

Cibacron® Blue, has been used as a ligand for liquid chromatography and for successfully depleting albumin for nearly thirty years (Travis, J. et al. Biochem J 1976, 157, 301-306). These textile dye materials were further developed into kit products for convenient use. Representative companies that offer this type of product are Millipore (Montage™ Albumin Depletion Kit) and Bio-Rad Laboratories (Aurum). The binding interaction between blue dye and albumin is not based upon specific affinity. While relatively inexpensive, non-specific depletion of other proteins is the major weakness of this technology. Sigma-Aldrich, using a type of Proprietary Blue Matrix (ProteoPrep™ Blue Albumin Depletion Kit), claims low non-specific binding because it does not contain Cibacron® Blue.

### IgG-Removal Protein-Sorbent Products

After albumin, the second most abundant protein in serum or plasma is IgG. Many vendors supply Protein A or Protein G sorbents, bacterial proteins that specifically bind the Fc region of IgG (Kronvall G. et al. J Immunol. 1970, 104, 140-147). This class of products is distributed by Sigma, Bio-Rad, Agilent, Applied Biosystems and Amersham (see below). Though not yet extensively used in proteomic studies, recombinant Protein L was cloned from Peptostreptococcus magnus and distributed by Affitech AS (Oslo, Norway). This protein binds the kappa light chain of antibodies from many species without interfering with their antigen binding sites. BioSepra™, the Process Division of Ciphergen Biosystems, has developed MEP HYPERCEL, an alternative to Protein A or Protein G for process-scale purification of recombinant antibodies and antibody fragments from many species. These sorbents may also be used in applications for the capture and separation of IgG antibodies in plasma or serum.

### Polyclonal Goat IgG Antibody Products

This group of products applies polyclonal Goat IgG antibodies against different target proteins for immunoaffinity binding and separation. Representatives include Amersham Biosciences (Ettan™ Albumin and IgG Removal Kit), Bio-Rad (Aurum Serum Protein Mini Kit), Applied Biosystems (POROS® Affinity Depletion Products) and Agilent Technologies (Multiple Affinity Removal Systems, MARS). These products contain Protein A or G either as a sorbent for direct binding IgG in plasma or serum, or as a linker for conjugating the polyclonal goat capture antibodies to the microbead surface.

These polyclonal goat IgG capture antibodies are affinity-purified and coupled through Protein A conjugated to the solid phase matrix. Their capacity (˜2 mg HSA per ml packed bed volume) is typically lower than the blue dye-based products, but they have much greater specificity for human serum albumin. In addition, depending on the plasma/serum loading, percentage albumin removal is also higher than with dye-based products, yielding a substantially superior product. For many sensitive proteomics applications, such as Multidimensional Protein Identification Technology (MudPIT) (Washburn M. P. et al. Nat Biotechnol. 2001, 19, 242-247), reducing HSA levels from the most abundant protein before depletion (˜60%) to the second or third most abundant protein after depletion (˜6%) is insufficient for direct analyses because of the very high backgrounds still evident.

The same point applies for IgG removal reagents, where >99% removal is ideal because of the immunoglobulins' very high initial abundance and their great molecular heterogeneity. Agilent's MARS use Protein A or Protein G for this purpose. Amersham and Bio-Rad offer prepacked spin columns that are convenient to use and only require a centrifuge and standard reagents and collection tubes. Agilent offers the most advanced application of IgG-based solutions by packing an HPLC column with POROS® 20 beads coupled to goat IgGs specific to HSA, IgG, Transferrin, α1-Antitrypsin, Haptoglobin and IgA. Standard LC fittings are provided. The user must have an LC system available to use Agilent's MARS columns. Complete kits are available, including two proprietary buffers, and filtration and concentration spin columns. About 85% of human serum proteins are removed, with very high-level capture of the target proteins. The columns are extensively reusable if handled properly. One drawback to this system is that urea is added to the extraction buffer which precipitates at low temperatures, requiring room temperature protein concentration for analyzing bound material.

Divide and conquer is a strategy that has been articulated to cope with the overwhelming dynamic range of protein concentrations present in proteomes, which is usually several orders of magnitude beyond the detection range of the currently most useful protein separation and identification methods, such as 2DE, LC and MS. For analytical management, the human proteome must be divided into sub-proteomes, and a complex protein mixture must be fractionated, in order to accurately separate and measure target proteins. Affinity separation is one of the most specific and effective approaches for fractionation of protein mixture or complex biological solution. Table 1 provides an overview, summarizing representative technologies and products.

TABLE 1
Technology and Product Comparison Summary

| Comparison | Technology 1 | Technology 2 | Technology 3 | Technology 4 | Technology 5 |
|---|---|---|---|---|---|
| Name and | Aurum ™ | ProteoPrep ™ | Albumin and | Multiple Affinity | IgY Microbeads of the |
| Features of | Serum Protein | Blue Albumin | IgG Removal | Removal System | Present Invention |
| Technologies | Mini Kit | Depletion Kit | Kit | | |
| Antibodies | none | none | Goat IgG | Goat IgG against | Avian IgY against HSA, |
| | | | against HSA | HSA, IgG, | IgG, IgA, IgM, |
| | | | | Transferrin, α1- | Transferrin, Fibrinogen, |
| | | | | Antitrypsin, IgA, | Haptoglobin, ApoA-I, |
| | | | | Haptoglobin | ApoA-II, α1-Antitrypsin, |
| | | | | | α1-Acid Glycoprotein, |
| | | | | | α2-Macroglobulin |
| Ligands | Cibacron Blue | Blue Dye and | Protein A or G | Protein A | No Protein A or G |
| | and Protein A | Protein G | | | |
| Microbeads | Resin | Agarose | Resin | POROS ® 20 | UltraLink Hydrazide Gel |
| Product Form | Spin Column | Spin Column | Spin Column kit | Pre-Packed HPLC | Microbead Slurry; Spin |
| | kit | kit | | Column kit | Column kits, and pre- |
| | | | | | packed FPLC columns |
| Capacity | 50 μl | 75 μl | 10-15 μl | 20-100 μl, | 10-175 μl, depending on |
| (Serum or | | | | depending on | target(s) |
| Plasma per ml | | | | column types and | |
| of product) | | | | sizes | |
| Target Protein | <90% | 80-95% | >95% | 98-99% | 95-99.5% |
| Removal | | | | | |
| Unique | No antibodies | Proprietary | Good albumin | Multiple reusable | Diversified product |
| Features | | Blue Matrix | specificity | | types, directly applicable |
| | | | | | to animals, mouse, rat, |
| | | | | | dog, etc. |
| Pros | Inexpensive, | Inexpensive, | Inexpensive, | Well-developed and | Convenient and specific |
| | convenient to | better | convenient to | having multiple | removal of 12 abundant |
| | use | specificity | use | target capacity. New | serum proteins in one |
| | | than Cibacron | | column available | step. >20-fold |
| | | Blue, low | | against mouse | enhancement of low |
| | | sample | | Albumin, IgG & | abundant proteins. All |
| | | dilution | | Transferrin. | antibody microbead |
| | | | | | products available |
| | | | | | separately. |
| Cons | Incomplete | Incomplete | Low capacity | Proprietary buffers. | Relatively lower capacity |
| | capture at | capture at | | Some IgGs | as MIXED12 in spin |
| | specified | specified | | unsuitable for | column format |
| | capacity. High | capacity | | column recycling, | |
| | non-specific | | | e.g. against ApoA-I, | |
| | protein capture | | | IgM, and α2- | |
| | | | | Macroglobulin | |

The present invention has a great potential for use on other body fluids, subcellular fractions, tissue and cell culture extracts, and other sub-proteomes. The technology is readily adaptable to different formats and scales of protein separation by using suitable devices or carriers. The unique biochemical and immunological features of this type of material enable its further development. The present invention can also be combined with other protein fractionation products to better meet the needs of scientists and provide solutions to facilitate protein target discovery and validation.

### BRIEF SUMMARY OF THE INVENTION

Briefly, in a specific embodiment of the present invention, an affinity separation composition is provided which comprises affinity reagents linked to a solid support and the methods that the solid support mediates affinity reagents to separate targets from non-targets in mixtures of biological samples. A preferred embodiment of the affinity reagents employed in the present invention is a polyclonal antibody composition of Immunoglobulin Yolk (IgY antibody) having an Fc region and an Fab antigen binding regions. The IgY antibody composition comprises a solid support covalently linked to oxidized glycosylation moieties in the Fc region of the polyclonal IgY antibodies wherein the Fab regions of the IgY polyclonal antibodies are capable of reacting with an antigen. The present invention also includes the above described polyclonal IgY antibody composition that additionally contains an antigen bound or hybridized to the Fab antigen binding regions of the antibody.

The present invention additionally includes a method of preparing the polyclonal IgY antibody compositions which comprises contacting reactive polyclonal IgY antibodies, wherein the glycosylation moieties in the Fc region have been oxidized, with a solid support material containing reactive moieties wherein the oxidized glycosylation moieties of the polyclonal IgY antibodies covalently bond with chemically reactive moieties of the solid support material by forming covalent bonds whereby the IgY polyclonal antibodies are oriented to allow the Fab regions to react with an antigen.

The reactive polyclonal IgY antibodies can be prepared using single or multiple antigens (immunogens). The antigens can be in a natural status of complex or isolated to highly (>99%) pure from the complex. The antibodies against single antigens can be mixed in a certain ratio before they are linked to solid support. Alternatively, the antibodies against single antigens can be linked to the solid support separately, then mixed in certain required ratio. The antibodies against multiple antigens can be conjugated to a solid support directly.

The present affinity separation compositions are used as affinity binding reagents to capture, separate, and detect one or more targets (proteins, antigens, or other biological materials) from a complex mixture. Using IgY antibody composition as an example, this affinity separation process can be generally accomplished by:
a. providing a complex target (protein or antigen) mixture;
b. contacting the complex target (protein or antigen) mixture with the present IgY polyclonal composition of the present invention whereby a desired target in the complex mixture binds with the IgY polyclonal antibodies in the Fab regions; and
c. recovering the treated complex target (protein or antigen) mixture wherein the concentration of the desired target (protein or antigen) has been substantially reduced for depletion or substantially enriched for affinity separation.

The complex target (protein or antigen) mixture can be plasma, serum, cerebrospinal fluid, urine, pulmonary alveolar lavage, vitreous humor, nipple aspirates, tissue samples, cell extracts or industrial streams from cell cultures. Additionally, the desired target (protein or antigen) that has specifically bound to the affinity reagents can be recovered and studied or analyzed to determine if other targets (protein or antigen) or compounds (e.g., lipids, hormones, etc.) in the complex are associated with the desired target.

Of particular interest in practicing the present invention, the major proteins present in serum are immunodepleted by contacting the serum with the present polyclonal IgY composition wherein the polyclonal IgY antibody is reactive with a major protein present in the serum. For example, human serum albumin (HSA) and IgG constitute approximately 75% of all proteins present in human serum. To eliminate HSA from serum, the serum would be contacted with the present polyclonal IgY antibody composition that contains anti-HSA IgY antibodies covalently conjugated to a solid surface, such as microbead carriers. Similarly, to eliminate IgG from the serum, the serum would be contacted with the present polyclonal IgY antibody composition that contains anti-IgG antibodies. Elimination of the predominant proteins is desirable because it makes detection and analysis of function of other proteins present in minor amounts easier in the depleted serum.

To effectively eliminate multiple protein targets through one step process (e.g. one IgY-microbead column), specific IgY microbead compositions can be prepared by combining the individual polyclonal IgY antibodies in a certain ratio and conjugating the mixed IgY antibodies to microbeads or by mixing individual IgY microbead preparation in certain ratio. For best capturing the target proteins or antigens, the ratio for mixing antibodies together is optimized based upon the relative quantity of the target proteins or antigens in a complex biological solution such as plasma or serum. The mixing ratio also takes into account of the various avidities of individual IgY to corresponding target protein or antigen.

The polyclonal IgY compositions of the present invention, directed against Albumin, IgG, Transferrin, α1-Antitrypsin, IgA, IgM, α2-Macroglobulin, Haptoglobin, Apolipoproteins A-I and A-II, Orosomucoid (α1-Acid Glycoprotein) or Fibrinogen, have all of the following advantages cited earlier:
High specificity for their targets;
High antigen-binding capacity (avidity) compared to other antibody-based products;
The same reagents are often applicable to multiple-species. Anti-human protein IgY antibodies often have a broad host range, with excellent binding of orthologous proteins from other mammalian species compared to IgG antibodies raised in rabbits, mice or goats, due to the great evolutionary distance between chickens and mammals;
Results are highly reproducible;
The compositions have good reusability; they can be recycled with little or no loss of antigen-binding specificity or capacity even after more than 20 uses;
There is minimal disruption to the natural condition of biological samples;
The compositions are convenient to use in a variety of formats, including preparative-scale Liquid Chromatography (LC) columns, spin columns, packed plugs in small tips, magnetic or paramagnetic micro- or nano-particles, or microfluidics devices;
The costs are reasonable, with the products generally affordable;
The materials can be made in large quantities due to efficiencies of production.

In another aspect of the present invention, the present polyclonal IgY antibody composition is made with anti-Fibrinogen IgY antibodies and this composition is used to deplete Fibrinogen (Coagulation Factor 1) from plasma. This will allow for proteomic analysis of the plasma proteins without the extensive proteolysis induced by standard methods of clotting. Thus, plasma proteomics analyses can be carried out with much greater precision than previously possible. The present polyclonal IgY antibody compositions can be employed to affinity-deplete high-abundant plasma and serum proteins that are present at levels above 1.0 mg/ml. Removing high-abundant proteins will enable researchers to effectively analyze low-abundance plasma proteins. This is particularly significant for detecting extremely low-level proteins at early disease stage, those induced by various drug treatments, toxicity detection, and for conducting multiplex protein profiling.

The polyclonal IgY compositions of the present invention can be used in high-throughput sample processing equipment, such as Applied Biosystem's BioCad Vision system. For example, see “Novel Plasma Protein Separation Strategy Using Multiple Avian IgY Antibodies For Proteomic Analysis”, in Methods in Proteomics (Smejkal, G., ed. 1994), which is incorporated herein by reference. This format is widely used by industrial-scale proteomics companies and has sophisticated, computer controlled sample handling capabilities with adjustable flow rates, various sized cartridge volumes, in-line pH monitoring and elution profiles.

### BRIEF DESCRIPTION OF THE FIGURES

FIG. 1—Basic composition and process of affinity separation. Listed are the various elements, components and materials that can be used to enable the composition and process of affinity separation of specific targets from mixture containing non-specific targets.

FIG. 2—Variations of basic compositions of affinity separation. Diagrams depict two examples of variations of the basic composition and process shown in FIG. 1. A, shown is to use the molecular affinity bridge, e.g. biotin and avidin or streptavidin, to link affinity reagent to solid support. B, multiple affinity reagents (e.g. IgY antibodies) mixed in certain ratio first, then linked to solid support, different from that in FIG. 1, where one affinity reagent linked to solid support.

FIG. 3—Comparison of one-round versus two-round depletion of HSA (FIG. 3 illustrates the depletion efficiencies using two sequential columns.)

FIG. 4—Initial capacity measurement of anti-HSA Microbeads. SDS PAGE analysis of HSA depletion in human serum samples. A: 4 μl serum; B: 10 μl serum.

FIG. 5—Repeated capacity measurement of anti-HSA Microbeads. 25 μl of diluted human serum (1:10 dilution in TBS to obtain concentration of 8 mg proteins/ml or 1:5 dilution in TBS to obtain concentration of 16 mg proteins/ml) were subjected to 2 rounds of HSA depletion by means of 25 μl IgY microbeads (conjugation ratio: 5 mg IgY/ml microbeads). A. HSA was completely removed from serum. The bound proteins are mainly albumin. Results shown are 3 μl/lane of pooled materials from 4 experiments. B. HSA was completely removed in 1:10 diluted serum, and 90% depletion was observed when serum was 1:5 diluted.

FIG. 6—Depletion of IgG by IgY Microbeads. Shows results wherein 50% of IgG-Fc was depleted for the samples at protein concentrations of 5 mg/ml. 80% depletion was observed for the samples at 2.5 mg/ml and 1.25 mg/ml. The negative control unconjugated microbeads failed to bind to IgG-Fc.

FIG. 7—Depletion of Apolipoprotein A-I by IgY Microbeads. Shows results wherein about 95% of Apolipoprotein A-I was depleted after 4 rounds of depletion.

FIG. 8—Protein Separation Capacity of IgY Microbeads for HSA. Shows the capacity of anti-HSA microbeads to be approximately 2.4 mg of HSA bound per ml of packed bed volume.

FIG. 9—Separation of Individual Target Proteins by IgY Microbeads. Shows the sequential depletion of four human proteins using individual IgY microbead gels, with virtually no cross-reactivity between non-targeted abundant proteins.

FIG. 10—Test of Separation Efficiency of MIXED6. The two-spin column system effectively removes all 6 target proteins (HSA, IgG, Fibrinogen, Transferrin, IgA and IgM) from human plasma.

FIG. 11A—Depletion of Human Plasma Using MIXED12. The one-spin column system effectively removes all 12 target proteins (HSA, IgG, Fibrinogen, Transferrin, IgA, IgM, Apolipoprotein A-I, Apolipoprotein A-II, Haptoglobin, α1-antitrypsin, α1-Acid Glycoprotein and α2-Macroglobulin) from two different pooled human plasma samples.

FIG. 11B—Depletion of Human Serum Samples Using MIXED12. The one-spin column system effectively removes all 12 target proteins (HSA, IgG, Fibrinogen, Transferrin, IgA, IgM, Apolipoprotein A-I, Apolipoprotein A-II, Haptoglobin, α1-antitrypsin, α1-Acid Glycoprotein and α2-Macroglobulin) from three different human clinical serum samples.

FIG. 12—Dimensional Electrophoresis of Human Serum Sample Treated by MIXED12. Direct evidence is provided for effective removal of the targeted abundant proteins in human serum.

FIG. 13—Dimensional Electrophoresis of Human Plasma Sample Treated by MIXED12. Direct evidence is provided for effective removal of the targeted abundant proteins in human plasma.

FIG. 14—Analysis of Recyclability of IgY Microbeads. Shows recycling of anti-HSA twenty times with no loss of capacity or specificity.

FIG. 15—Analysis of Recyclability of MIXED12 Spin Column. Shows recycling of MIXED12 twenty times with no loss of capacity or specificity.

FIG. 16—Serial Depletion of 8 Mouse Plasma Proteins by IgY Microbeads. Shows effective sequential depletion of at least 7 of the 8 orthologous mouse proteins using anti-human protein IgY microbeads.

FIG. 17—Comparison of anti-HSA and Anti-BSA IgY Microbeads. Panel A: anti-HSA IgY microbeads; Panel B: anti-BSA IgY microbeads. Consistent with Table 5, significant differences are shown between the cross-species albumin binding capacities (human, bovine, mouse and rat) of anti-HSA IgY microbeads and anti-BSA IgY microbeads.

FIG. 18—Analysis of depletion effects of IgY microbeads against HSA, IgG and Transferrin, when certain specific ratios of the antibodies were mixed based on the relatively quantity of the target proteins (HSA, IgG and Transferrin) in human plasma as well as their avidity to the targets.

### BRIEF DESCRIPTION OF THE TABLES

Table 1—Technology and Product Comparison Summary

Table 2—Efficiency of anti-HSA IgY microbeads for “spiked” samples in PBS using different antibody loading densities on microbeads

Table 3—MIXED IgY Microbead Products

Table 4—Depletion Efficiency of MIXED12 after Multiple Cycles

Table 5—Depletion Efficiency of Anti-HSA and BSA IgY Microbeads

Table 6—Example Ratio of Mixing IgY Microbeads against HAS and IgG

Table 7—Example Ratio of Mixing IgY Microbeads against HAS, IgG, and Transferrin

### DETAILED DESCRIPTION OF THE INVENTION

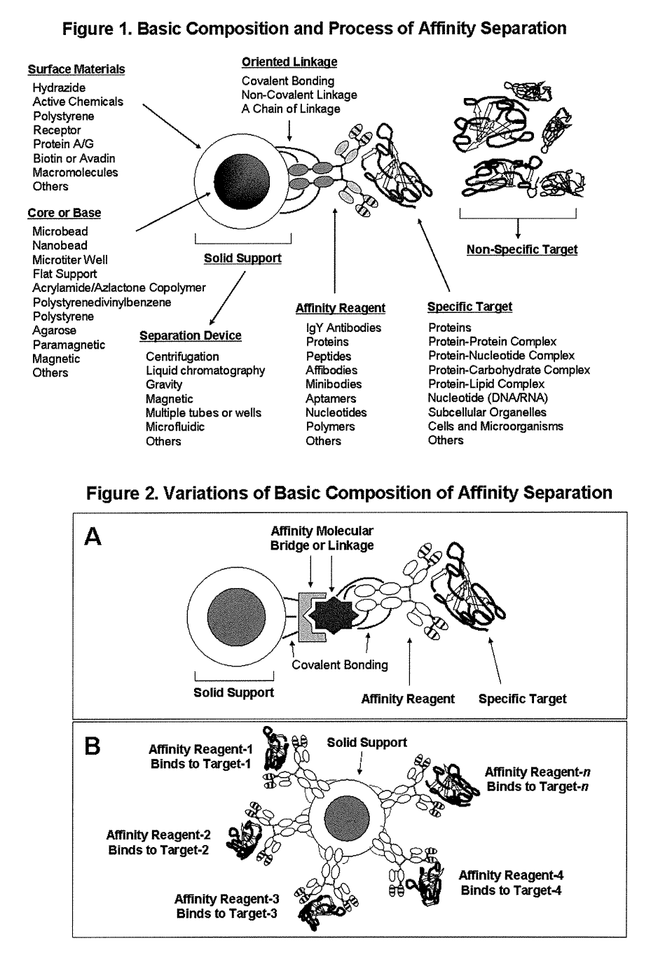

In practicing the present invention, the following basic components, processes, and variations (FIGS. 1 and 2) are employed to conduct an affinity separation process:

Affinity Reagent(s)—These are biological substances or macromolecules that can specifically bind to targets through affinity recognition and attractive forces between reagents and targets. Affinity recognition, resembling the relationship between lock and key, is highly specific for the target and usually has a dissociation constant below 10⁻⁸ M Winzor D. J., J Chromatogr. 2004 1037(1-2): 351-67; Chaiken I. M., J Chromatogr. 1986, 376: 11-32). The affinity reagents can include IgY antibodies, proteins, peptides, affibodies, minibodies, aptamers, nucleotides, polymers and others.

Specific Targets or Target(s)—These are also biological materials, macromolecules, molecules, or complexes. The specific targets are usually antigens that can induce antibodies in animals. The specific targets can also be other materials such as proteins, protein-protein complexes, protein-nucleotide complexes, protein-carbohydrate complexes, protein-lipid complexes, nucleotide (DNA/RNA), subcellular organelles, cells and microorganisms and others. The specific targets are usually mixed or complexed with other non-specific targets. The specific targets that bind specifically to affinity reagents can be separated from those non-specific targets in a given mixture of specific targets and non-specific targets.

Oriented Linkage or Orientedly Linked or Orientedly Coupled—These are the chemical or biological materials that can link affinity reagents to the surface of solid supports. Linkages can be covalently bonding between the affinity reagents and the surface of the solid support. Linkages also can be indirect, through a chain of covalent bonding and non-covalent affinity binding, as shown in FIG. 2. In particular, affinity reagents can be orientedly coupled or linked to a solid support such that the affinity reagents are interacted with the support in a manner as to facilitate the activity of the affinity reagents (e.g., increasing the binding activity or efficiency of the affinity reagents to the corresponding targets).

Solid Support—These are the materials that are attached to the affinity reagents through oriented linkage and can mediate the affinity reagents to separate bound targets from those non-specific targets. The solid support generally comprises surface materials and a core or base. The surface materials are the active chemical or biological materials that can link the solid support to the affinity reagents. These materials comprise hydrazide, active chemicals, polystyrene, receptor, protein A/G, biotin, avidin, strepavidin, macromolecules and others. The core or base is coated with the surface materials and linked to affinity reagents via surface materials. The core or base can be the materials that help or mediate the separation of that affinity reagent-target complex. Examples of the core or base include microbeads, nanobeads, microtiter wells, flat supports, acrylamide/azlactone copolymer, polystyrenedivinylbenzene, polystyrene, agarose, paramagnetic, magnetic and others.

Separation Devices—These are the forces, attractions, apparatus, or processes that mediate the separation of the affinity reagent-target bound solid support from mixture of targets or biological materials. Examples of the separation devices include gravity, centrifugation, liquid chromatography, magnetic force, multiple tubes or wells, microfluidic and others.

The basic composition and process of affinity separation specified in the present invention are depicted in FIG. 1 with some examples of related materials. The composition and process can be engineered into different variations. FIG. 2 depicts two classes of variations:

Variation 1—Shown in FIG. 2A, the linkage of affinity reagents to solid support is indirect, which is designed to have a molecular bridge, a pair of affinity reagents such as biotin and avidin. Each end of the molecular bridge is fixed to the affinity reagent or solid support through covalently bonding. This is a type of chain linkage, where the linkers can be combinations of covalent or non-covalent associations.

Variation 2—The solid support can be attached to affinity reagents in a different way. Shown in FIG. 2B, the solid support is bound to a group of affinity reagents mixed at a given ratio before the linkage process takes place. The ratio of mixing of affinity reagents is based upon the optimized binding of the affinity reagents to targets and effectiveness of affinity separation.

Immobilized Affinity Reagent—An immobilized affinity Reagent is formed when an affinity reagent is or a plurality of affinity reagents are linked or coupled (e.g., orientedly linked to coupled) to a solid support. A plurality of the affinity reagents may be homogenously mixed from the same source of affinity reagents (e.g, the affinity reagents which bind to the same target) or heterogeneously mixed from various sources of affinity reagents (e.g., various affinity reagents which bind to different, various targets). The affinity reagent may be covalently linked to the solid support or non-covalently linked to the solid support through molecular affinity bridge (e.g., non-covalent bonding between two molecules) as shown in FIG. 2(A). The immobilized affinity reagent may be capable of binding to a target through the affinity reagent which binds to the target.

In one embodiment of the present invention, an affinity separation composition for separating one or more target compounds present in a complex mixture is made by linking an affinity reagent to a solid support oriented in a manner to facilitate the activity of the affinity reagents or its ability to further react with a target. Once prepared the affinity separation composition is contacted with a complex mixture to remove the target from the mixture by affinity recognition of the target by the affinity reagent. The solid support component of the affinity separation composition mediates the separation of the affinity reagent-target complex from the complex mixture. The resulting complex mixture has a reduced level of the target and preferably no detectable levels of the target. The affinity reagent-target complex can then be processed to strip the target so that the affinity separation composition can be re-used. Additionally, the target can be recovered and/or analyzed to determine if there is an association between other materials and the target.

More specifically, in practicing the present invention, polyclonal IgY antibodies can be covalently conjugated to a solid support material by oxidizing the glycosylation moieties in the Fc region of the polyclonal IgY antibodies and then reacting oxidized antibodies with a solid support material that has reactive moieties that will form a covalent bond (conjugation) with the oxidized glycosylation moieties. This reaction forms an antibody composition that orients the antigen binding region away from the support material and allows the antibody to react with an antigen.

While the exact ratio of IgY antibody to solid support material is not critical, typically 5, 10, 15, or 20 mg of IgY antibody are reacted with 1 ml of solid support material. The solid support material can be of any desired shape, size or physical configuration such as microbeads, membranes, chip surfaces and the like. Any shape having a large surface area is preferred. The chemistries involving the oxidation and conjugation reactions are well known to one of ordinary skill in the art.

The solid support may contain a spacer arm that reduces steric hindrance and allows the orientation of the antibody so that the Fc region is positioned toward the support and the Fab regions are positioned away from the support where it can more readily reactibind with an antigen. A support material and spacer arm with minimal nonspecific binding characteristics is preferred. The specific length of the spacer arm is not critical and spacer arms can be up to 23 atoms or longer if desired.

The solid support can be in any physical configuration such as for example beads or membranes. However, any configuration that increases the surface area of the solid support is preferred because an increased surface area will allow for more attachment sites of the IgY antibody in a given volume. For this reason beads, including nanobeads, are a preferred solid support configuration. Beads can be in a pre-packed or batch mixture format. Beads can also be used in a continuous process format. Magnetic and paramagnetic beads can be also be employed as the solid support to aid in the separation of the polyclonal IgY beads after being contacted with the complex protein mixture being immunodepleted.

If a solid support material is used that will react specifically with the Fab regions of the IgY antibody then the support material can be coated to render the material non-reactive to the Fab regions and facilitate a reaction with the Fc region of the IgY antibody. For example, polystyrene beads, including styrene nanobeads, can be coated with avidin or streptavidin to prevent reactions between the polystyrene and the Fab regions on the antibody. The avidin coated polystyrene beads are then reacted with biotin that has been modified to contain hydrazide groups that can then react with the Fc region of the IgY antibody. This allows for the proper orientation of the IgY antibody for maximum efficiency in hybridizing with the desired protein (antigen) in the complex protein mixture. In another example, periodate-oxidized IgY is reacted with a bifunctional linker molecule containing a hydrazide at one end and a ligand at the other end. The resulting IgY-ligand molecule then binds tightly and specifically with a ligand receptor bound to a solid surface, such as a microbead. The linker molecule is bifunctional and comprises an hydrazide moiety at one end to bind to the Fc region of the IgY and biotin at the other end which serves as the ligand to bind to the solid support. Coupling of the biotinylated IgY to a solid surface is mediated through avidin or streptavidin which coats the underlying solid surface.

Once the polyclonal IgY antibody composition (collectively referred to hereinafter as “present IgY composition”) is prepared, it can be used to immunoprecipitate a desired protein from a complex protein mixture. This is done by contacting or incubating a sample of the complex protein mixture with the present IgY composition. The depleted sample can be recovered and contacted with a fresh or recycled batch of the present IgY composition one or more additional times depending on the binding capacity and protein concentration of the sample. The sample is then analyzed to determine if all of the desired protein has been removed from the sample. Additionally, after the depletion is complete, the present IgY composition used in the depletion reaction can be treated to strip the desired protein from the antibodies, which can then be analyzed to determine if other proteins or materials are associated with the desired protein.

The exact amount of polyclonal IgY antibody composition used in practicing the present invention (immunodepletion process) is not critical as any available IgY antibody will react with the target protein. Excess amounts of IgY antibody are employed if all of the target protein is to be removed from the complex protein mixture. If less than all of the target protein is to be removed from the complex protein mixture then the amount of IgY antibody is adjusted accordingly. Routine titration experiments can be conducted to determine the optimum amount of antibody needed per weight of target protein.

In human serum depletion with the present polyclonal IgY composition it is desirable to remove at least about 95% by weight and preferably at least about 98% of the high abundant proteins. For HSA removal at least about 99% and preferably at least about 99.9% or more is removed from the complex protein mixture.

Another aspect of the present invention relates to an immunoaffinity separation composition for separating or depleting a plurality of targets in a complex mixture or enriching non-targets in the complex mixture comprising a first immobilized affinity reagent capable of binding to a first target and a second immobilized affinity reagent capable of binding to a second target, wherein the first immobilized affinity reagent comprises a first affinity reagent being linked to a first solid support and the second immobilized affinity reagent comprises a second affinity reagent being linked to a second solid support. The first affinity reagent is prepared using the first target and capable of binding to the first target; and the second affinity reagent is prepared using the second target and capable of binding to the second target. In one embodiment, a mixture ratio between the first immobilized affinity reagent and the second immobilized affinity reagent or between the first affinity reagent and the second affinity reagent can be determined so as to efficiently reduce the targets in the mixture complex (e.g., substantially deplete the amount of the targets in the mixture).

In another embodiment, the first solid support is different or separate from the second support. The first affinity reagent is linked to the first solid support to form a first immobilized affinity reagent. In parallel, the second affinity reagent is linked to the second solid support to form a second immobilized affinity reagent. The first immobilized affinity reagent and the second immobilized reagent are then mixed together at a mixture ratio to form the composition.

In another embodiment, the first solid support and second solid support are the same support. The first affinity reagent and the second affinity reagent are mixed in a mixture ratio. The mixed reagents are then linked to the solid support.

In another embodiment, the ratio of the first and second targets in the complex mixture can be determined through methods known in the art, including but not limited to, SDS gels, ELISA, Western blots, HPLC, and Luminex. In one embodiment, the ratio is a weight ratio. In a preferred embodiment, the ratio is a molar ratio.

In another embodiment, the immunoaffinity separation composition further comprises a third immobilized reagent capable of binding to a third target, a forth immobilized reagent capable of binding to a fourth target, a fifth immobilized reagent capable of binding to a fifth target, a sixth immobilized reagent capable of binding to a sixth target, and/or an nth immobilized reagent capable of binding to an nth fourth target (n can be 7, 8, 9, 10, 11, 12, 13, 14, 15, 16, 17, 18, 19, 20, 50, 100, and more).

In another embodiment, the mixture ratio of the affinity reagents or the immobilized affinity reagents can be determined as to efficiently reduce the amount of targets or separate the targets from the mixture with a desired reduction efficiency. The desired reduction efficiency is the removal or separation efficiency of a target or targets in a mixture after the mixture is contacted with the separation composition. For example, the desired reduction/separation efficiency can be >about 75%, >about 80%, >about 85%, >about 90%, >about 95%, >about 99%, or between about 80% to about 100% (or about 99%), about 90% to about 100% (or about 99%), about 95% to about 100% (or about 99%) removal or sepratation of the target or targets from the mixture. In order to efficiently reduce/separate targets at the desired efficiency or efficiencies, factors to be considered include, but are not limited to, the density of the affinity reagent(s) on a solid support, the affinity level or capacity of the affinity reagent(s) or the immobilized affinity reagent(s) with the corresponding target(s) (in case of an antibody, the avidity of the antibody), the interaction between or among the (immobilized) affinity reagents once mixed, and the ratio of the targets in the mixture complex. Once desired reduction efficiencies are set for a plurality of targets respectively (e.g., 95% reduction efficiency for the first target and 99% reduction efficiency for the second target), the capacity of the first and second affinity reagents (or the first and second immobilized affinity reagents) are measured through determining the amount of each individual target which can be removed for each unit of the reagents. The ratio of the first target and the second target in the mixture complex can be used approximately or substantially as a starting ratio and adjusted to a first testing ratio based on the capacity of the reagents or immobilized reagents. For example, the ratio is measured at 4 (the first target) to 1 (the second target) and the capacity of the first immobilized reagent is higher. In this case, the starting ratio 4:1 may be adjusted to a first testing ratio 3.5:1 as less first reagents may be used. The first and second reagents (or immobilized reagents) are then contacted with the mixture and the flow-through fraction is analyzed to determine whether the desired reduction or separation efficiencies for the first and second targets are reached. If not, the first testing ratio can be incrementally readjusted by increasing or decreasing a small percentage (e.g., 1%-20%, about 5%, or about 10%) of the first and/or second reagents (or immobilized reagents) (e.g., changing the first testing ratio of 3.5:1 to a second testing ratio of 3.3:1.2. The reagents or immobilized reagents are then mixed at the second testing ratio and contacted with the mixture complex. The adjustment can be repeated until the desired reduction or separation efficiencies are approximately or substantially reached in the flow-flow through fraction.

In another embodiment, the composition can be used to reduce or separate targets or enrich non-targets in the complex mixture through the steps of contacting the complex mixture with the immunoaffinity separation composition and collecting a flow-through. A flow-through is a resultant mixture after the complex mixture is contacted with the composition wherein the concentration of the targets, to which the composition is capable of binding, in the flow-through has been reduced compared to that in the complex mixture. The composition can also be used to enrich the targets or separate the targets from non-targets in the complex mixture through steps of contacting the complex mixture with the composition and collecting an elution fraction. The elution fraction is the elution or recovery of the targets which bind to the composition after the complex mixture is contacted with the composition wherein the concentration of non-targets in the elution fraction has been reduced compared to that in the complex mixture.

In a specific embodiment, the affinity reagents are IgY antibodies and targets are proteins. When mixtures of different IgY antibodies are used to substantially deplete multiple target proteins from a complex mixtures to achieve >about 99% reduction efficiency, the ratio of the different IgY antibodies correlates approximately to the ratio of the target proteins present in the complex protein mixture but is adjusted by the capacity of individual IgY antibody. Capacity of individual IgY microbeads is defined as the amount of the target antigen in human serum/plasma efficiently depleted by per ml of corresponding IgY microbeads.

The reduction efficiency (the percentage of the removed target antigen) for individual IgY microbeads is set based on the amount of target antigen in biological samples such as serum/plasma and avidity of its IgY, ranging from >95% to >99%. For example, about 50% of total human serum proteins is albumin, the reduction efficiency for anti-HSA IgY microbeads is set more than 99% because 1% of residual albumin (0.5%) in flow-through fraction is still in abundant protein range interfering the down stream potential biomarker analysis. Due to the variation of avidity for individual polyclonal IgY generated by immunizing different antigens in chickens, it is preferred to determine the capacity of individual IgY microbeads experimentally. For a typical capacity assay for individual IgY microbeads, different amount of human serum/plasma containing the target antigen are incubated with a fixed amount of the individual IgY microbeads in a spin column, respectively. The flow-through fractions (unbound) are analyzed by both SDS-PAGE to observe if the target antigen band disappeared in the fractions and ELISA to determine the reduction efficiency by measuring the percentage of the residual target antigen in the flow-through fractions. Furthermore, in order to obtain a high capacity for individual IgY microbeads, different amount of IgY are coupled to the microbeads to optimize the coupling density for individual IgY microbeads such as 10 mg IgY/ml beads, 15 mg IgY/ml beads and 25 mg IgY/ml beads, then capacity is tested for each fixed density using different amount of the target antigen. It is observed that that highest coupling density (say 25 mg IgY/ml beads) may not necessarily give the highest capacity, especially for large target antigens like IgG, IgA, IgM and 2α-Macroglobulin. Most likely, the interaction between IgYs on microbeads and large antigen could be easily saturated due to the steric hindrance of large size of the target antigen.

Each type of mixture is initially formulated based on the amount of target antigen in the serum/plasma and the determined capacities of individual IgY microbeads. Due to complex interactions of the target antigens in serum/plasma, formulated each type of IgY microbeads mixture needs to be tested for the capacity. Capacity assay is similar to that used for individual IgY microbeads. However, multiple target antigens will be analyzed by SDS-PAGE and ELISA. It is important to incrementally adjust the ratio of individual IgY microbeads components in the mixture while still keep the mixture volume the same based on the result of analysis. The purpose for fine adjustments of individual IgY microbead is to have an optimal capacity while reduction efficiencies for the target antigens meet the desired percentages.

As mentioned above, routine analytical procedures (SDS gel, ELISA, Western blot, etc.) are employed to determine the ratio of target proteins present in the complex protein mixture and then the corresponding IgY antibody ratios are calculated and mixed accordingly. For example, if HSA and IgG are the target proteins in a serum sample and upon analysis of the serum sample are present in a weight ratio of 3:1 (HSA/IgG, 50% vs 17%), or molar ratio about 6.6:1 (MW of IgG is 2.2 times as that of HSA) then it would be preferred to employ an IgY antibody composition that contain about 87% anti-HSA IgY antibodies and 13% anti-IgG IgY antibodies (always use molar ratio of target antigens to adjust the ratio of mixed IgY microbeads, 6.6:1 ratio) in amounts effective to reduce of the target proteins present in the complex mixture.

The 6.6:1 ratio between the two antibodies can be further adjusted or fine-tuned according to the methods in the present invention for any given reduction efficiency to be reached. After adjustment, the final ratio for the two antibodies was set for 4:1 (see detailed description in Example 15). If other target proteins are to be removed from the serum then the ratios of all of the target proteins are calculated and the specific IgY antibodies are prepared in accordance to the calculated protein ratios.

The following terms are defined for use herein:

“IgY polyclonal antibody” means gamma globulins derived from the egg yolk of an avian species.

“Avian species” refers to any bird, preferably chickens (Gallus gallus).

“Covalently linked” when referring to IgY antibodies means oriented conjugation of the IgY antibodies with the antigen binding fragment available for antigen binding. This occurs by oxidizing the IgY-Fc glycosylation moieties, converting hydroxyl groups to reactive aldehyde groups, which then react with chemical groups on the solid support forming stable covalent bonds.

“Antigen” means any compound that is recognized and specifically bound by the polyclonal antibody preparation. Typically, this same antigen is used to immunize the bird for producing polyclonal antibodies in the yolk. The immunization is typically done by injecting a bird with a purified antigen. In the case of protein antigens, a bird can be injected with polynucleotides that can express the protein antigen or immunogenic portions thereof thereby making the antigen in situ in the bird.

The present invention is particularly useful in depleting abundant proteins present in plasma, serum and other body fluids and tissue samples to allow for a more accurate quantitation of less abundant proteins present in those materials. Abundant proteins present in serum include, but are not necessarily limited to, human serum albumin (HSA), IgG, Transferrin, IgA, α2-Macroglobulin, IgM, α1-Antitrypsin, Complement C3, Haptoglobin, Apolipoprotein A-I, Apolipoprotein A-II, Apolipoprotein B, and α1-acid glycoprotein (Orosomucoid). In addition to these highly abundant proteins, plasma also contains Fibrinogen and other clotting factors.

To deplete serum or plasma of any one or more of these abundant proteins, polyclonal IgY compositions of the present invention are prepared using an antibody that will hybridize to the desired protein to be depleted. The serum or plasma sample is contacted with that specific IgY composition to remove the desired protein. Preferably, all proteins present in plasma and serum in an amount of 1.0 mg/ml or greater are immunodepleted according to the present invention. The process can be repeated to remove additional proteins. Alternatively, two or more antigen specific polyclonal IgY compositions can be combined and then several proteins can be depleted in a one step process.

Other applications of the present IgY polyclonal antibody compositions include their use in IgY antibody arrays, IgY antibody microbeads that will hybridize with any desired protein whether it is an abundant protein or not, IgY antibody columns and IgY antibody diagnostic applications.

IgY antibodies are made in birds and preferably chickens. The birds are injected with the purified protein (desired protein to be removed from the complex protein mixture) that acts as an antigen in the bird resulting in the production of IgY antibodies that will bind with the protein. This produces high affinity antibodies with high avidity. Gene-specific IgY antibodies can also be made by injecting gene expression vectors where the antigenic protein is made in situ. IgY is then collected from the yolks of bird eggs employing standard separation techniques. See Drug Discovery Today, Vol 8, No 8, 2003, 364-371, which is incorporated herein by reference.

The IgY antibodies specific for the desired protein are separated from the other IgYs by antigen affinity purification employing similar procedures to the antigen affinity purification of IgG. See The Journal of Cell Biology, Volume 141, Number 7, Jun. 29, 1998 pp. 1515-1527, which is incorporated herein by reference. For affinity purification of anti-HSA IgY, purified HSA is coupled to cyanogen bromide-activated Sepharose 4B (Pharmacia Biotechnology, Inc.). The Total IgY preparation is then contacted with the HSA-bound Sepharose 4B, wherein the anti-HSA antibodies specifically bind to the antigen on the Sepharose beads. After washing to remove the non-specific IgY antibodies, the anti-HSA antibodies are then eluted sequentially with 0.1 M glycine-HCl (pH 2.5) and the column neutralized with 0.1 M triethylamine (pH 11.5) before reequilibration. The affinity purified IgY antibodies are then used in a reaction with the reactive solid support material to make the present polyclonal IgY compositions.

In one embodiment, the purified IgY antibodies are oxidized in the Fc glycosylation region with sodium metaperiodate, followed by dialysis to remove the oxidizer. The oxidized IgY antibodies are then reacted with azlactone-acrylamide copolymer microbeads (Pierce UltraLink® Hydrazide Gel), which is an affinity support for immobilizing glycoproteins through oxidized sugar groups. A preferred bead diameter is in the range of from about 50 to about 80 μm with an average diameter of about 60 μm. It is ideal for immobilizing IgY polyclonal antibodies since they contain abundant carbohydrates located on the Fc portion of the antibody molecule. Because such antibodies are coupled to the UltraLink® Hydrazide Gel through the Fc portion only, they are properly oriented with their antigen-binding sites unobstructed, offering greater antigen binding capacity. In order to optimize orientation of the antigen binding region for optimal antigen binding capacity the following spacer arm is employed with the azlactone-acrylamide copolymer microbeads:

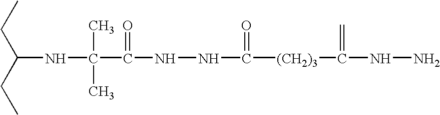

The immobilization chemistry uses sodium periodate to oxidize glycoproteins, converting vicinal hydroxyl groups in sugars to reactive aldehyde groups. The aldehydes then react with hydrazide groups on the UltraLink® Hydrazide Gel to form stable hydrazone bonds. The coupling conditions are flexible with regard to time and temperature. A long (23-atom) spacer arm that reduces steric hindrance and a support with minimal nonspecific binding characteristics makes this a favorable gel for affinity chromatography. The protein-coupled columns may be regenerated and reused at least 20 times under the proper stripping and regeneration conditions.

UltraLink® Biosupport Medium is hydrophilic, charge-free, high-capacity, highly cross-linked, rigid, copolymeric and porous. This means that the support has minimal nonspecific interactions with the sample. The porosity, rigidity and durability of this support are important considerations when working with large volumes of samples requiring fast-flow techniques and large-scale applications. Agarose supports are extremely useful for gravity flow procedures; however; a more rigid support is required if pressures are greater than 25 psi. UltraLink® Biosupport Medium is useful for medium-pressure techniques. When packed into a 3 mm inside diameter×14 cm height column, UltraLink® Supports have been run to approximately 400 psi (system pressure) with no visual compression of the gel or adverse effects on chromatography. Typically these columns can be used with linear flow rates of 85-3,000 cm/hour with excellent separation characteristics. See, for example, Brown, M. A., et al. (2000). Identification and purification of vitamin K-dependent proteins and peptides with monoclonal antibodies specific for gamma-carboxyglutamyl (Gla) residues. J. Biol. Chem. 275(26), 19795-19802; Coleman, P. L., et al. (1988). Affinity chromatography on a novel support: azlactone-acrylamide copolymer beads. FASEB J. 2: A1770 (#8563); Coleman, P. L., et al. (1990). Azlactone copolymer beads: applications in bioseparations. J. Cell. Biochem. 44, 19 (S14D); Milbrath, D. S., et al. (1990). Azlactone-functional supports useful in affinity chromatography and other bioseparations. AIChE Extended Abstracts #104E; Milbrath, D. S., et al. (1989). Azlactone polymer supports for bioseparations. ACS Abstracts; Rasmussen, J. K., et al. (1991/1992). Crosslinked, hydrophilic, azlactone-functional polymeric beads: a two-step approach. React. Polym. 16, 199-212; Rasmussen, J. K., et al. (1992). Mechanistic studies in reverse-phase suspension copolymerization of vinyldimethylazlactone methylenebis (acrylamide). Makromol. Chem., Macromol. Symp. 54/55, 535-550; Rasmussen, J. K., et al. (1990). Hydrophilic, crosslinked, azlactone-functional beads- a new reactive support. Polymer Reprints 31(2), 442-443; U.S. Pat. No. 4,871,824 (Heilmann, et al.); and European Patent Publication 0 392,735 A2 all of which are incorporated herein by reference.

Serum protein depletion can be achieved by loading 50 μl of anti-HSA IgY azlactone-acrylamide copolymer microbead slurry (25 μl beads) onto a Handee Mini-spin Column (Pierce, Prod #69705) and inserting the column in an Eppendorf tube, which is centrifuged for 8 seconds at full speed to remove the solution. Then 25 μl of serum (recommended 6- to 10-fold dilution of serum) is added to the dried microbeads and incubated at room temperature for 30 min. The microbeads should be resuspended once every 5 minutes with gentle stirring using a Pipetman tip. After incubation, the column is inserted into a clean Eppendorf tube and centrifuged for 8 seconds at full speed. The collected sample is subjected to another round of depletion as described above. The obtained sample is ready for further study and/or further depletion of another protein, such as IgG, employing a specific anti-IgY covalently conjugated to azlactone-acrylamide copolymer microbeads. Likewise other proteins can be depleted if desired.

In another embodiment of the present invention, the protein that is immunoprecipitated onto the polyclonal IgY compositions of the present invention can be analyzed to determine if there is an association between the immunopreciptated protein and any other proteins or other compounds, such as lipids, carbohydrates, hormones and the like, present in the serum. To analyze protein bound to IgY microbeads, the microbeads are washed 2× with 0.5 ml TBS and then eluted with 25 μl of 100 mM glycine-HCl pH 2.5. The collected sample is then neutralized with 2.5 μl of 1M Tris-base pH 8 and is then ready for analysis.

Following are examples that illustrate procedures for practicing the invention. These examples should not be construed as limiting its scope. All percentages are by weight and all solvent mixture proportions are by volume unless otherwise noted.

### EXAMPLES OF THE INVENTION

### Example 1

### Direct Covalent Conjugation of Individual IgY Antibodies to Solid Support

IgY microbeads were initially developed by optimizing conditions for covalently conjugating affinity-purified anti human serum albumin (HSA) IgY to UltraLink® Hydrazide Microbeads (Pierce Biotechnology, Rockford, Ill., USA) at different antibody-microbead conjugation ratios and by optimizing the conditions of HSA depletion using anti-HSA IgY-microbeads in a “batch” mode. Affinity-purified anti-HSA IgY antibodies (3 mg/ml) were oxidized with sodium meta-periodate (5 mg/ml), at room temperature for 30 minutes, followed by dialysis against 4 L of Phosphate Buffered Saline (PBS), in a 2 ml/dialysis cassette (Pierce Product No. 66425: Slide-A-Lyzer Dialysis Cassettes, 10 k MWCO) at 4° C. for 1 h, with 3 changes of buffer. Oxidized IgY was incubated with Hydrazide microbeads (Pierce Product No. 53149) to obtain conjugation ratios of 5, 10, 15 and 20 mg IgY/ml microbeads. Conjugation was carried out at 4° C. overnight with rotation. After conjugation, microbeads were washed with 1M NaCl and followed by 3× with PBS, and stored as a 50% slurry in PBS.

### Example 2

### Test of Depletion Efficiency of Anti-HSA IgY Microbeads Using Purified Human Serum Albumin

Titration of the binding efficiency of anti-HSA IgY microbeads were carried out using Handee Mini-Spin Column (Pierce Product No. 69705) and HSA-spiked PBS samples. Fifty microliters (50 μl) of 50% microbeads were centrifuged (8 seconds) in a spin column. Dried microbeads were quickly incubated with 25 μl samples containing 0.72, 1.39, 2.72, 7.35, 10.85 or 14.83 mg/ml HSA (Diagnostic Grade) (US Biological, Product No. A1327-15) in PBS measured by BCA protein assay. These represented total amounts of 18 μg, 35 μg, 68 μg, 184 μg, 271 μg and 371 μg protein, respectively. Binding reactions were performed in the column at room temperature for at least 30 minutes. IgY microbeads were gently resuspended once every 3-5 minutes using disposable pipette tips. After incubation, the column was inserted into an Eppendorf tube and centrifuged for 8 seconds at 14,000 rpm in a microfuge. Proteins in collected samples were quantified by BCA. Table 2 summarizes the experimental results, using different ratios of IgY microbead to target protein concentrations. These results were obtained with one-round of depletion, in most cases using quadruplicate samples.

TABLE 2
Efficiency of Anti-HSA IgY Microbeads for “Spiked” Samples in PBS

| | HSA Depletion Efficiency (%) | | | | | |
|---|---|---|---|---|---|---|
| IgY/Bead | 18 μg | 35 μg | 68 μg | 184 μg | 271 μg | 371 μg |
| (mg/ml) | (0.7 mg/ml) | (1.4 mg/ml) | (2.7 mg/ml) | (7.4 mg/ml) | (11 mg/ml) | (15 mg/ml) |
| 5 | 100 | 100 | 85 | 59 | n.t. | n.t. |
| 10 | 100 | 100 | 100 | 52 | 38 | 35 |
| 15 | n.t. | n.t. | n.t. | 61 | 43 | 47 |
| 20 | n.t. | n.t. | n.t. | 51 | 49 | 15 |

n.t.: not tested.

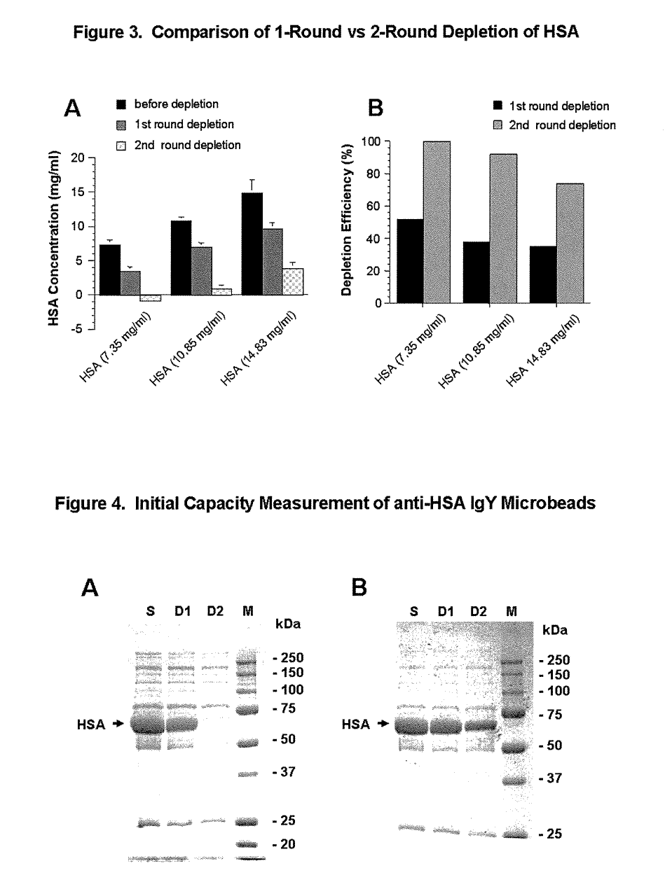

Titration was further carried out through a process of two serial rounds of depletion for removal of additional HSA. “10×” microbeads (=10 mg IgY/ml microbeads) were mixed with 25 μl of 7.35, 10.85 and 14.83 mg/ml HSA, equivalent to 184, 271 and 371 μg protein, respectively. Flow-through samples from 1^{st} round depletion were collected and subjected to a 2^{nd} round of depletion with the identical amount of fresh microbeads (FIG. 3).

### Example 3

### Depletion of Human Serum Albumin (HSA) from Serum Samples using Anti-HSA IgY Microbeads

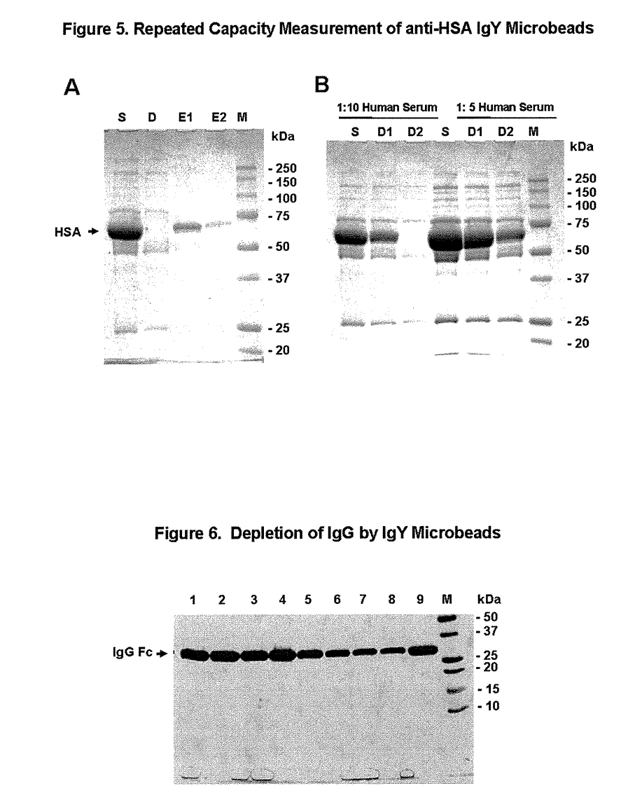

To test HSA depletion in a human (male) serum sample (Sigma, H-1388, Lot 122K0424), either 4 μl or 10 μl human serum samples were diluted to a total of 25 μl in PBS. Two rounds of depletion were performed using “10×” microbeads (=10 mg IgY/ml microbeads) as described in Example 2. To analyze depletion results, 2 μl of collected sample were diluted to 20 μl in sample loading buffer and boiled for 3 min. After cooling, 15 μl (for 4 μl serum depletion) or 5 μl (for 10 μl serum depletion) samples were subjected to 10% SDS-PAGE, followed by Coomassie Blue R-250 staining (FIG. 4, A, 4 μl serum depletion; B, 10 μl serum depletion). Tests of depletion of HSA from human serum samples were repeated. Twenty-five microliter (25 μl) of diluted human serum (1:5 and 1:10 dilution in TBS) were subjected to 2 rounds of HSA depletion using 25 μl “5×” IgY microbeads (conjugation ratio: 5 mg IgY/ml microbeads). Results shown in FIGS. 5, panel A was with 3 μl/lane of pooled materials from 4 experiments (serum dilution at 1:5). Lane D in panel A shows that all of the HSA was removed completely from the serum after 2 rounds of depletion. FIG. 5, panel B shows the results of each depletion from human serum diluted 1:5 or 1:10. As clearly depicted in the picture, HSA was completely removed by the 5× microbeads in 1:10 diluted serum, and about 90% depletion was observed when serum was diluted 1:5 (Panel B, Lanes D2). The microbead elution lanes (Panel A, lanes E1 and E2) show that HSA was the only protein removed from the serum. This elution fraction can be analyzed by proteomics techniques well known in the art, such as 2-dimensional gel electrophoresis and mass spectrometry, to sensitively analyze other proteins co-purifying with HSA. The use of two anti-HSA columns in series avoids the need for substantial sample dilution. Using this technique with 25 μl microbead (50 μl slurry) volumes in a batch mode, HSA was almost completely removed from 4 μl serum diluted 6-fold, and about 65% of the HSA was removed from 10 μl serum diluted 2.5-fold, in both cases without any noticeable loss of other proteins (data not shown).

### Example 4

### Depletion of Human Immunoglobulin

Affinity-purified anti-IgG-Fc IgY antibodies were covalently conjugated to UltraLink Hydrazide Microbeads using the method described in Example 1. Fifty microliter (50 μl) of purified human IgG-Fc (Calbiochem Catalog No. 401104) was spiked into PBS solution at concentrations of 10, 5, 2.5 or 1.25 mg/ml. In a control sample, human IgG-Fc (50 μl of 1.25 mg/ml, unoxidized) was spiked to PBS and incubated with unconjugated microbeads. The samples were subjected to one-round of depletion with anti-IgG-Fc-microbeads, by separation in a Handee Mini-Spin Column (Pierce Product No. 69705). The depleted samples were collected. Both starting materials (before depletion) and collected samples were diluted 10-fold (Lanes 1, 5), 5-fold (Lanes 2, 6), 2.5-fold (Lanes 3, 7) and 1.25-fold (Lanes 4, 8, and 9) to obtain a final concentration of 1 mg/ml, followed by SDS PAGE analysis. FIG. 6 shows that about 50% of IgG-Fc was depleted for the samples at a protein concentration of 5 mg/ml. 80% depletion was observed for the samples at 2.5 mg/ml and 1.25 mg/ml. The negative control unconjugated microbeads failed to bind to IgG-Fc (Lane 9).

### Example 5

### Depletion of Human Apolipoprotein A-I

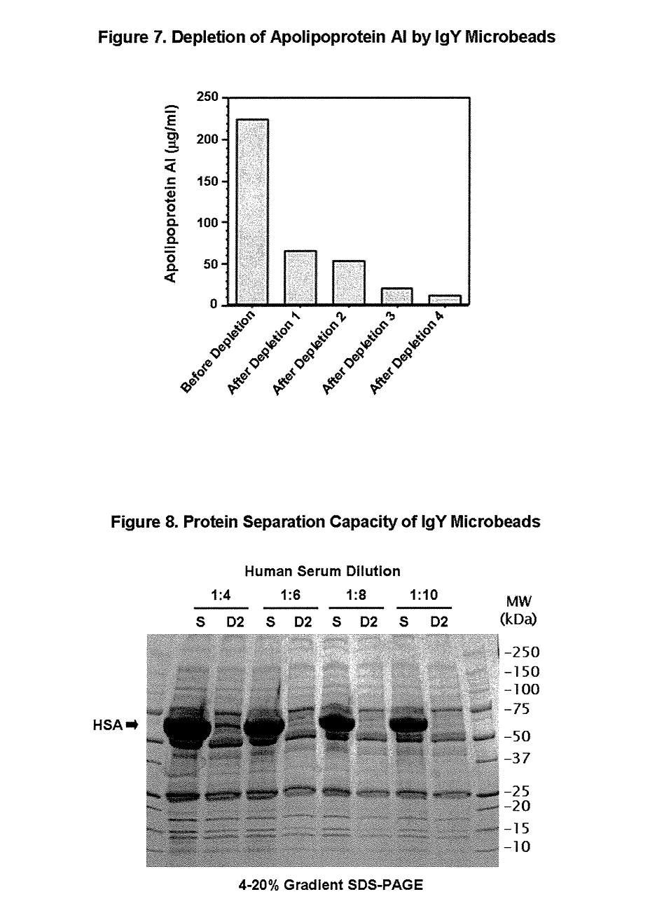

Affinity-purified anti-Apolipoprotein A-I IgY antibodies were covalently conjugated to CarboLink Agarose Beads (Pierce Biotechnology) essentially using the method described in Example 1. One hundred microliters (100 μl) of purified human Apolipoprotein A-I (Calbiochem Catalog No. 178452) was spiked into PBS solution at a final concentration of 0.225 mg/ml. The sample was sequentially subjected to four-rounds of depletion with anti-Apolipoprotein A-I-beads in a Handee Mini-Spin Column (Pierce Product No. 69705). The depleted samples from each round were collected, and subjected to BCA protein analysis. FIG. 7 shows about 95% of Apolipoprotein A-I was depleted after 4 rounds of depletion.

### Example 6

### Capacity of IgY Composition in Immunoaffinity Separation of Abundant Proteins from Non-Abundant Proteins in Serum/Plasma Samples

The binding capacity of IgY microbeads varies with different IgY antibodies against corresponding target proteins, and is related to the natural concentration of the target protein in serum/plasma, and to the avidity of the IgY antibody for its target, and to the concentration of capture antibody on the solid surface. To empirically test the capacity of anti-HSA IgY microbeads, human serum samples were diluted with TBS (Tris-Buffered Saline, 10 mM Tris-HCl, 0.15 M NaCl, pH 7.4) at ratios of 1:4, 1:6, 1:8, and 1:10. Equal volumes (25 μl) of the bed volume of microbeads and the diluted human serum samples (S) were mixed and incubated. The IgY microbeads were separated from the solution with a spin column device. The unbound materials (flow-through solution or 1^{st} fraction of depletion) were further mixed and incubated with another fresh 25 μl bed volume of IgY microbeads to repeat the separation process. This resulted in the 2^{nd} fraction of depletion (D2). The untreated diluted serum samples (S) and the 2^{nd} fraction of depletion (D2) were resolved on a 4-20% gradient SDS-PAGE under reducing conditions and were visualized via Coomassie Blue staining. As shown in FIG. 8, anti-HSA IgY microbeads completely depleted HSA from diluted serum in 2×25 μl batches if the serum is diluted greater than 4-fold. S: Starting, unfractionated human serum. D2: Unbound material after 2 rounds of anti-HSA depletion. Assuming 40 mg/ml HSA in undiluted serum, capacity is equivalent to ˜2.7 mg HSA captured/ml microbeads, where 1 ml microbeads contain about 10 mg IgY antibodies.

### Example 7

### Specificity of IgY Composition in Immunoaffinity Separation of Abundant Proteins from Non-Abundant Proteins in Serum/Plasma Samples

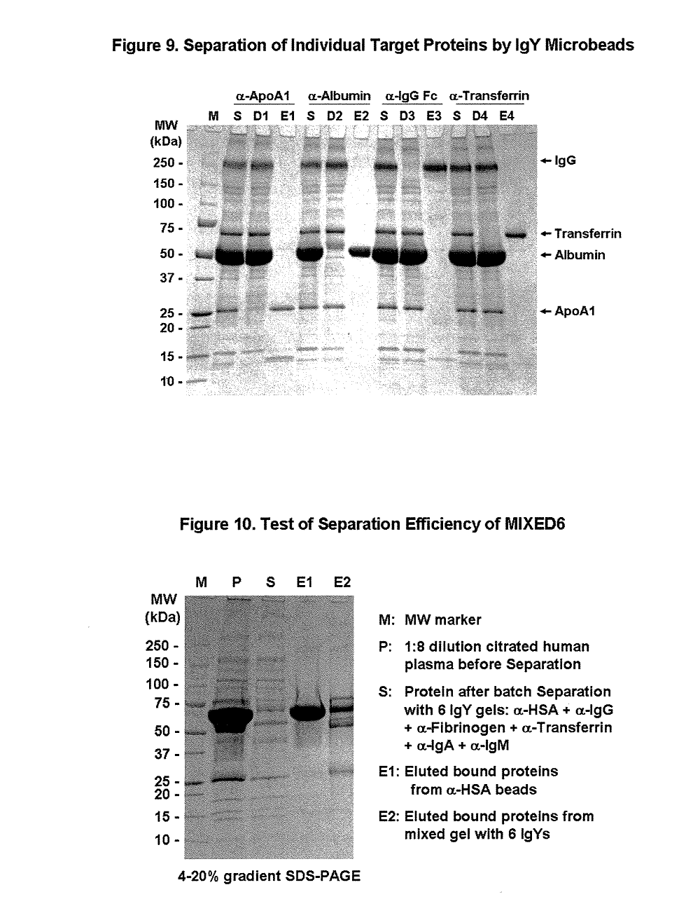

A critically important feature for any proteomics sample preparation composition or method is the specificity of capture of the target protein. Indeed, antibodies are among the most specific capture reagents available. To test the specificity of IgY microbeads for their intended targets, human serum samples were diluted in Tris-Buffered Saline (TBS dilution buffer, 10 mM Tris-HCl, 0.15 M NaCl, pH 7.4) based on abundance of the target protein, added to pre-packed IgY-microbead spin columns using empty Micro Bio-Spin Columns (Cat. No. 732-6204) and End-Caps (Cat. No. 731-1660) (Bio-Rad, Hercules, Calif., USA), and incubated at room temperature for 15 minutes with rotation. The samples depleted of the target proteins (flow-through) were collected in a 2 ml microcentrifuge tube by centrifugation (at 5,000×g for 15 seconds in a microcentrifuge). The spin columns were washed three times with TBS containing 0.05% Tween-20 (wash buffer) to remove residual unbound proteins, then the bound proteins were twice eluted with 0.1M Glycine, pH 2.5 (stripping buffer). For each elution, IgY microbeads in the spin column were mixed and incubated with the stripping buffer at room temperature for 3 minutes followed by centrifugation to collect the eluted proteins. After elution, the spin columns were immediately neutralized with 0.1 M Tris-HCl, pH 8.0, and pooled eluted fractions were neutralized with 1/10 volume 1 M Tris-HCl pH 8.0 (neutralizing buffer). Unfractionated samples, depleted fractions, and eluted-bound protein fractions were analyzed by 1-DE. A few representative examples are shown in FIG. 9. Albumin, IgG, and Transferrin are three highly abundant proteins in serum and plasma. Apolipoprotein A-I is the most abundant lipoprotein. Compared to unfractionated samples, Apolipoprotein A-I, Albumin, IgG, and Transferrin were effectively removed in flow-through samples (FIG. 9, lanes D1, D2, D3, and D4). Proteins bound to the corresponding IgY columns were predominantly the expected targets (FIG. 9, E1, E2, E3, and E4). Despite albumin being such a dominant protein in the serum, representing about half of the total protein mass, other proteins with far less abundance were effectively and specifically removed in the presence of albumin. These results demonstrate that IgY microbeads can efficiently and specifically separate complex serum proteins.

### Example 8

### Efficiency of IgY Composition in Immunoaffinity Separation of Abundant Proteins from Non-Abundant Proteins in Serum/Plasma Samples

There is an unmet need to simultaneously remove multiple abundant plasma proteins. To determine the efficiency of simultaneous removal of several of the most abundant plasma proteins, individual IgY microbead compositions were mixed at an optimized ratio based on the relative abundance of their target proteins and avidity of IgY antibodies. Two types of mixed IgY microbeads, MIXED6 and MIXED12, were produced. The key features of two types of MIXED IgY microbeads are summarized in Table 3.

TABLE 3
MIXED IgY Microbead Products

| Products | MIXED6 | MIXED12 |
|---|---|---|
| IgY Antibody Targets | Albumin | Albumin |
| | IgG | IgG |
| | Transferrin | Transferrin |
| | Fibrinogen | Fibrinogen |
| | IgA | IgA |
| | IgM | IgM |
| | | α1-Antitrypsin |
| | | α2-Macroglobulin, |
| | | Haptoglobin, |
| | | Apolipoprotein A-I |
| | | Apolipoprotein A-II, α1- |
| | | Acid Glycoprotein |
| Spin Column Process | Two-step process | One-step process |
| Separation Efficiency | Remove about 88% | Remove about 95% total |
| | total plasma proteins | plasma proteins |

Human plasma samples were diluted and treated with MIXED6 IgY microbeads through a two-step spin column process, with the flow-through from the first anti-HSA antibody spin column then being passed through a spin column filled with the appropriate mixture of anti-HSA and 5 other microbead-coupled IgY antibodies. FIG. 10 shows the results of removal of six abundant plasma proteins using MIXED6. M, Molecular weight marker; P, 1:8 dilution citrated human plasma before depletion; D, P after depletion with MIXED6 IgY microbeads; E1, Eluted bound proteins from anti-HSA microbeads; E2, Eluted bound proteins from MIXED6 IgY microbeads. Proteins were visualized by 4-20% gradient SDS-PAGE with Coomassie Blue staining.

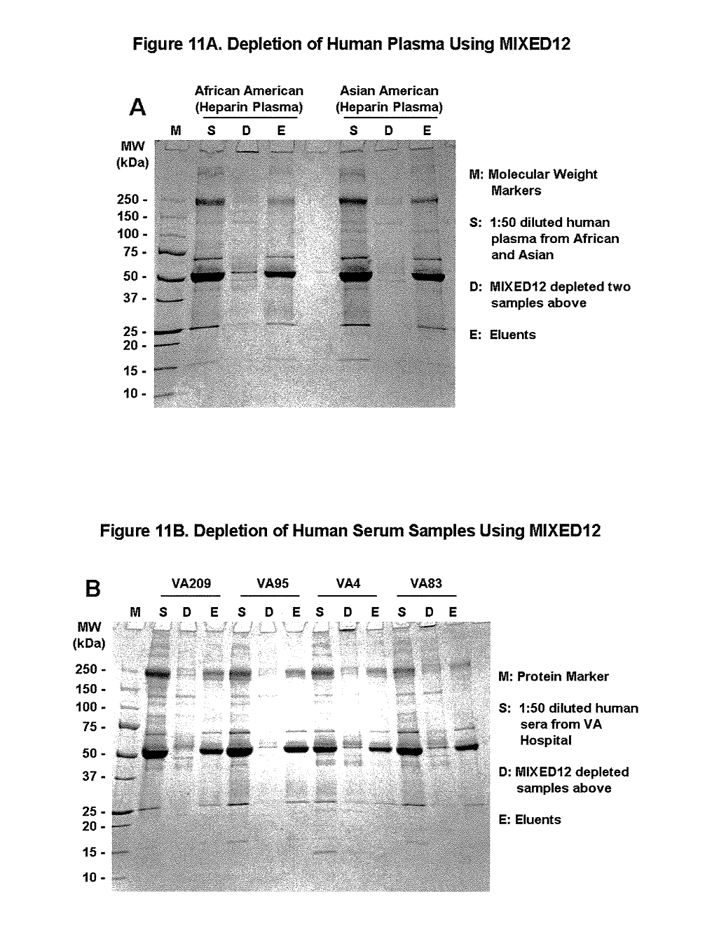

To enhance the convenience of use, a one-column system was employed for MIXED12. Plasma or serum samples were treated with MIXED12 spin columns essentially as described in Example 7. Two representative examples are shown in FIG. 11A and 11B. Six sets of three fractions were loaded onto 4-20% SDS gel under non-reducing conditions: unfractionated samples before loading to spin columns (S), depleted of target proteins (D), and eluted-bound proteins (E). M is a molecular weight marker. Compared to unfractionated samples, the target proteins were effectively removed from the flow-through samples (Lanes D). Proteins bound to the corresponding IgY columns were mainly the expected targets (Lanes E). These results demonstrate that MIXED12 can efficiently and specifically separate complex serum proteins.

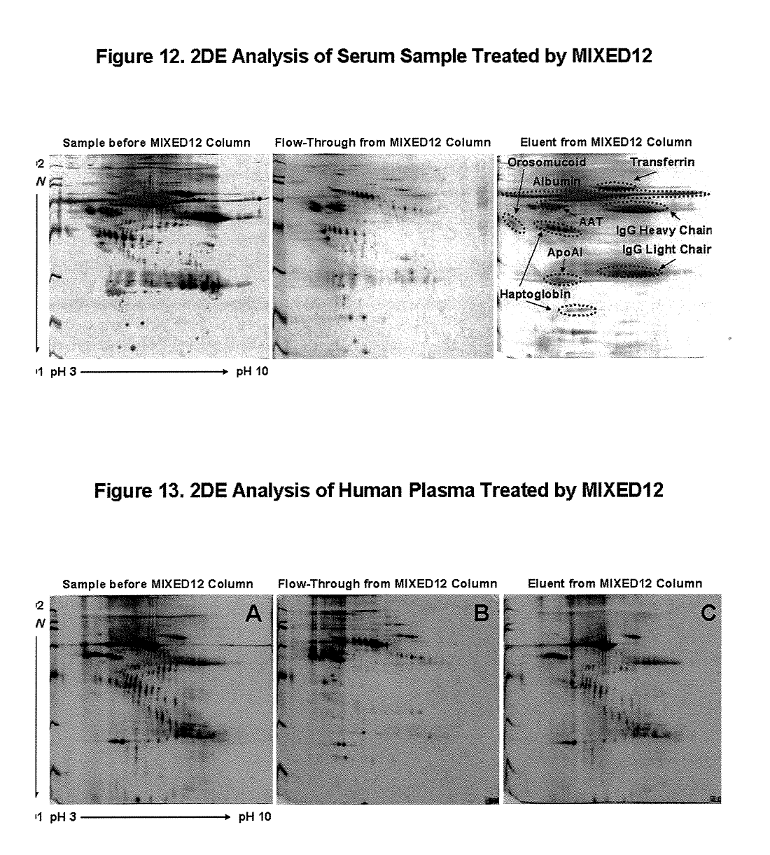

Plasma or serum samples treated with MIXED12 were further analyzed by two-dimensional gel electrophoresis (2DE). Three samples (unfractionated, depleted and eluted samples, ˜100 μg each) were precipitated using acetone and dissolved in a rehydration solution (9.5 M urea, 4% CHAPS, 18 mM DTT, 0.5% IPG buffer pH 3-10, trace of bromophenol blue), and loaded into a 13 cm long pH 3-10 NL Immobiline™ DryStrip (Amersham Biosciences). The DryStrip was laid on the sample solution, covered with paraffin oil and allowed to rehydrate overnight. IPGphor (Amersham Biosciences) was used for the first dimension IEF, for a total running time of 42,000 Vh. Prior to the second dimension separation, the strip was equilibrated (50 mM Tris-HCl pH 8.8, 6 M urea, 30% glycerol, 2% SDS, trace of bromophenol blue, 100 mg of DTT was added per 10 mL solution prior to use) for 15 minutes in a screw-cap culture tube followed by alkylation with lodoacetamide (25 mg/ml) for 15 minutes. Proteins were separated vertically by second-dimensional SDS-PAGE (12% acrylamide) at 10° C. and visualized by staining with Gel Code™ (Pierce Chemical Co.). The resulting 2DE images were analyzed by comparisons with standard human serum and plasma 2DE maps found in the pubic domain FIGS. 12 and 13. In comparison, many protein spots previously obliterated by abundant proteins were revealed in the flow-through, depleted fraction. Selective removal of highly-abundant proteins significantly improved the 2DE resolution of plasma proteins. The removed proteins were detected. Definitive protein identification can be carried out after cutting out the gel spots, followed with trypsin digestion and peptide mass fingerprinting using MALDI-TOF mass spectrometry analysis.

### Example 9

### Recyclability of IgY Composition and Reproducibility of Immunoaffinity Separation

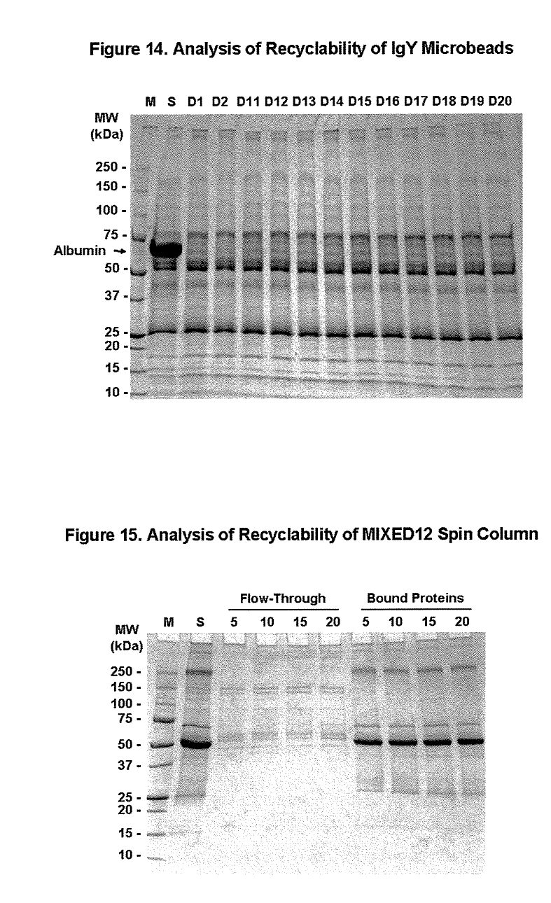

Reproducibility measures the accuracy that a product can perform through a repetitive process. Recyclability is an indication of the endurance of a product and its capability of being regenerated without loss of either capacity or specificity. To evaluate the reusability of the IgY microbead columns of the present invention, an important factor in their economic use, the depletion efficiency of IgY microbead columns over multiple cycles was systematically analyzed. First, anti-HSA IgY microbead spin column was used to separate proteins from aliquots of the same human serum sample 20 times in succession. FIG. 14 shows the selected samples analyzed by IDE 4-20% SDS PAGE under non-reducing conditions. M, Molecular weight marker; S, Sample of human serum sample; D1, D2, D11-20, Samples of flow-through from the corresponding cycle of the same anti-HSA IgY spin column. The fractions depleted of albumin from D1 to D20 are virtually identical, demonstrating high reproducibility and recyclability of anti-HSA IgY microbeads. MIXED12 product was also tested for its recyclability and reproducibility. In order to make the MIXED12 column reusable, bound proteins must be efficiently and completely removed without damaging the antibodies coupled to the column. In addition, all 12 targeted proteins must be eluted from the column under the same buffer conditions. First, individual IgY microbead columns with single protein targets were tested with serum or plasma samples running through multiple cycles. ELISA or Western blotting methods were used to evaluate the depletion efficiency by assaying residual proteins in the depleted fractions. Under identical binding, washing, stripping, neutralizing and reequilibrating buffer conditions, all 12 proteins were efficiently removed from their corresponding columns. Twenty aliquots of a human serum sample were sequentially run through cycles in the same MIXED12 spin column containing the same microbead composition. The flow-through and eluted fractions were collected. Selected fractions were analyzed by IDE, ELISA, and Western Blotting. As illustrated in FIG. 15, indistinguishable protein banding patterns were observed in samples collected at cycles 5, 10, 15, and 20, indicating high reproducibility with a single column over multiple cycles. The ELISA and Western blotting results for cycle #20 are summarized in Table 4. Among the twelve proteins, Albumin, IgG, IgA, Transferrin, α2-Macroglobulin, Apolipoprotein AI and AI, and Fibrinogen were reproducibly removed to near completion. Haptoglobin, α1-Antitrypsin, Orosomucoid, and IgM were also significantly removed, although with slightly less efficiency than the other eight proteins.

TABLE 4
Depletion Efficiency of MIXED12 after Multiple Cycles

| | Relative abundance in | | Method |
|---|---|---|---|
| Protein | serum (average %)^{a} | % Removal | of Detection |
| Albumin | 54 | >99.5% | ELISA |
| Immunoglobulin G | 17 | >99.5% | WB |
| Transferrin | 3.3 | >99.5% | WB |
| Haptoglobin | 3.0 | 92-95% | WB |
| α1-Antitrypsin | 3.8 | >95.0% | WB |
| α2-Macroglobulin | 3.6 | >99.5% | WB |
| Immunoglobulin A | 3.5 | >99.5% | WB |
| Immunoglobulin M | 2.0 | 90-95% | ELISA |
| Orosomucoid | 1.3 | 92-95% | WB |
| Apolipoprotein AI | 3.0 | >99.5% | WB |
| Apolipoprotein AII | 1.0 | >99.5% | WB |
| Fibrinogen | 3.0 (plasma)^{b} | >99.5%* | WB |

^{a and b}Approximate weight-based protein abundance value in normal serum [Putnam, F. R. 1984. The Plasma Proteins, vol. IV, Academic Press, Orlando, FL. and Tybjaerg-Hansen, A. B. et al. 1997. A common mutation (G-455--> A) in the beta-fibrinogen promoter is an independent predictor of plasma fibrinogen, but not of ischemic heart disease. A study of 9,127 individuals based on the Copenhagen City Heart Study. J Clin Invest. 99: 3034-3039].
*The data for Fibrinogen were obtained in a separate experiment using an individual antibody spin column and human plasma sample.

### Example 10

### Effectiveness of Anti-Human Protein IgY Composition in Immunoaffinity Separation of Orthologous Proteins from Plasma Samples of Other Mammals

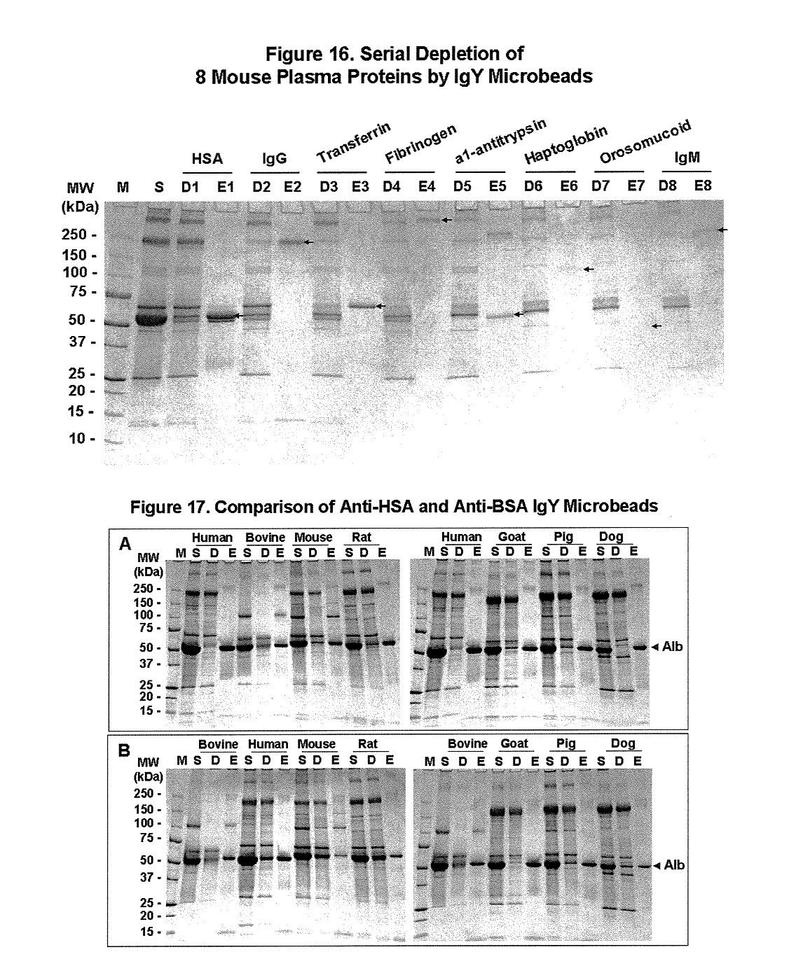

Due to the sequence similarity of many serum/plasma proteins between human and rodents, and the great evolutionary distance between birds and mammals, chicken antibodies against human proteins are likely to cross-react with their rodent orthologs. Mouse plasma samples were tested individually with several of the present IgY antibody microbead compositions against human plasma proteins in Western blot assays. Eight anti-human protein IgY antibody microbead compositions bound their corresponding mouse plasma proteins. To further confirm the Western blotting results, mouse plasma was sequentially run through eight IgY microbead columns, each with a different antibody. Collected fractions were analyzed by 1-dimensional SDS-PAGE (1DE) (FIG. 16). Specific protein depletion was clearly revealed, demonstrating that IgY antibodies directed against these human proteins, except Orosomucoid, can effectively be used to separate orthologous mouse plasma proteins. Removal of Orosomucoid was not detected by SDS-PAGE despite the fact that antibody cross-reactivity to the same protein of mouse and rat origin was confirmed by Western blot assay. In FIG. 16, M, Molecular weight marker; S, Unfractionated mouse plasma; D1, Plasma depleted of albumin; E1, Eluted-bound protein to anti-HSA IgY microbeads; D2, D1 depleted of IgG; E2, Eluted-bound protein to anti-IgG IgY microbeads; D3, D2 depleted of Transferrin; E3, Eluted-bound protein to anti-Transferrin IgY microbeads; D4, D3 depleted of Fibrinogen; E4, Eluted-bound protein to anti-Fibrinogen IgY microbeads; D5, D4 depleted of α1-antitrypsin; E5, Eluted-bound protein to anti-α1-Antitrypsin IgY microbeads; D6, D5 depleted of Haptoglobin; E6, Eluted-bound protein to anti-Haptoglobin IgY microbeads; D7, D6 depleted Orosomucoid; E7, Eluted-bound protein to anti-Orosomucoid IgY microbeads; D8, D7 depleted of IgM; E8, Eluted-bound protein to anti-IgM IgY microbeads. Arrows indicate the target proteins.

### Example 11

### Comparison Binding Specificity of Anti-HSA IgY Composition to Anti-BSA IgY Composition

As demonstrated in Example 10, at least seven IgY microbead compositions directed against human plasma proteins can effectively bind to orthologous mouse proteins. In addition, anti-HSA IgY cross-reacts to albumin in several different species, such as mouse, rat, pig, and goat. Bovine serum albumin (BSA) is also an abundant protein present in large amounts in many tissue culture media. To assess whether anti-BSA IgY has same binding capacity and cross-species host range as anti-HSA IgY, a comparison experiment was performed. Human, cow, mouse, rat, pig, goat and dog serum samples were diluted 1:20 in TBS. Hundred microliters (100 μl) of anti-HSA or anti-BSA IgY microbeads were mixed with 100 μl of each diluted serum sample in a spin column. After 15 minutes of incubation with rotation, the albumin-depleted fraction was removed by brief centrifugation. The beads were then washed three times with TBS. The bound albumin was eluted with stripping buffer (0.1M Glycine-HCl, pH 2.5). The eluted fraction was neutralized immediately with 1 M Tris-HCl buffer pH 8.0. Protein concentration was measured by the BCA method following supplier's instruction (Pierce). Table 5 shows the comparison of the binding capacity of anti-HSA IgY microbeads and anti-BSA IgY microbeads to albumins of other species in duplicated experiments.

TABLE 5
Depletion Efficiency of Anti-HSA and BSA IgY Microbeads

| | |
|---|---|
| Anti-HSA IgY | Anti-BSA IgY |

mg antigen bound to the microbeads

| Species | Test 1 | Test 2 | Test 1 | Test 2 |
|---|---|---|---|---|
| Human | 2.00 | 2.22 | 1.60 | 1.72 |
| Bovine | 1.12 | 1.12 | 1.38 | 1.45 |
| Mouse | 1.46 | 1.51 | 1.10 | 1.46 |
| Rat | 1.22 | 1.18 | 0.88 | 0.93 |
| Pig | 1.28 | 1.22 | 1.38 | 1.51 |
| Goat | 1.20 | 1.12 | 1.77 | 1.98 |
| Dog | 1.06 | 1.06 | 0.95 | 1.09 |

As shown in the table, anti-HSA IgY and anti-BSA IgY displayed quite distinct binding patterns to serum albumins of other mammalian species. Anti-HSA IgY has higher cross-reactivity to mouse and rat albumin, while anti-BSA IgY binds more goat and pig albumin. These different patterns were further illustrated in FIG. 17. Unfractionated, depleted and eluted fractions of each serum sample from Example 11, Test 1 were analyzed on ID SDS-PAGE. After treatment with anti-HSA IgY microbeads, albumin in human, mouse, rat, pig, and dog sera was completely or almost completely removed (Panel A, lanes D under corresponding species names). In contrast, anti-BSA IgY beads efficiently removed albumin only from bovine, goat, and pig sera. The majority of mouse and rat albumin was not captured by the anti-BSA IgY microbeads; these rodent albumins still remained in the flow-through fractions (Panel B, lanes D under corresponding species names). This finding is consistent with the differences noted in the quantitative binding study above, and confirms significant differences in cross-species albumin reactivity between anti-HSA IgY microbeads and anti-BSA IgY microbeads.

### Example 12

### Indirect Linkage of IgY Antibodies to Solid Support Via Alternative Affinity Binding Reagents: Biotin and Avidin or Streptavidin

Covalent coupling of IgY antibodies to solid support via a bifunctional hydrazide linkage is shown as an example for indirect linkage, and alternative strategy for coupling antigen affinity purified IgY antibodies to solid supports such as microbeads, nanoparticles, etc. Mild oxidation of IgY with sodium periodate will produce reactive aldehydes on the carbohydrate moieties of the Fc portion that then can be alkylated by hydrazides. This approach is advantageous for antibodies because they become covalently modified in a manner that maintains immunological reactivity, and it is ideal for polyclonal IgY antibodies because they are heavily glycosylated. The configurations of this chemistry are quite flexible and encompassed by the claims in this application. In addition to the method described in Example 1, where carbohydrates on the Fc portion of antigen affinity purified IgY oxidized by sodium metaperiodate were covalently linked to a hydrazide-coated microbead surface, other configurations are envisioned. For example, periodate-oxidized IgY is reacted with a bifunctional linker molecule containing a hydrazide at one end and a ligand at the other end. The resulting IgY-ligand molecule then binds tightly and specifically with a ligand receptor bound to a solid surface, such as a microbead.

Two specific examples are illustrated below (biotin/avidin and biotin/streptavidin). In these examples, the bifunctional linker molecule comprises hydrazide at one end and the ligand is biotin at the other end. Coupling of the biotinylated IgY to a solid surface is mediated through avidin or streptavidin. Coupling of Biotin-Hydrazide to IgY antibody is done according to the manufacturer's instructions: Pierce Biotechnologies (Product number 21340: EZ-Link™ Biotin Hydrazide, Spacer Arm: 15.7 Å, Molecular Weight: 258.34; or Product Number 21340 EZ-Link™ Biotin-LC-Hydrazide, Spacer Arm: 24.7 Å, Molecular Weight: 371.50).

After biotinylating the carbohydrates on the IgY antibodies, the molecules are reacted with a planar surface, microbeads or nanobeads coated with avidin or streptavidin. Examples of such solid support products include Dynabeads MyOne™ Streptavidin, Dynabeads® M-280 Streptavidin or Dynabeads® M-270 Streptavidin from Dynal Biotech (Brown Deer, Wis.), or Power-Bind™ Streptavidin Microparticles from Seradyn (Indianapolis, Ind.).

### Example 13

### Multiplex (96-Well Plate) Format of Application

IgY microbeads and IgY composition can also be applied to multi-well or array format apparatus, such as microplates having a membrane at the bottom of each well. Two hundred microliter (200 μL) of a slurry (50%) containing the MIXED12 IgY microbeads is aliquoted into a 96-well filter plate (Cat #F20036 or F20009 from Innovative Microplate, Mass., USA). To remove the buffer, the plate is centrifuged at 1,000 rpm for 1 minute in an Eppendorf bench top centrifuge with plate adapter. The separation process includes the following steps: Rinse/centrifuge the plate 2-3 times with 100 μL PBS. Discard the PBS rinse. Dilute 1 μL plasma in 99 μL of PBS, add to well, mix with pipette tip and incubate for 30 min at room temperature on a shaker. Centrifuge the plate as described above and collect Fraction 1 (˜100 μL) in NUNC cat #260251. Add 100 μL PBS/0.02% Tween-20 to the well. Centrifuge and collect Fraction 2 into fresh NUNC cat #260251. Add 100 μL 0.1 M glycine pH 2.5 to the well, centrifuge and collect Fraction 3. Add 100 μL 0.1 M Tris pH 8.0 to the well, centrifuge and add to fraction 3 (˜200 μL). The resulting Fraction 1, Fraction 2 and Fraction 3 are then analyzed using standard analytical techniques such as, for example, an H50 chip surface on a SELDI mass spectrometer (Ciphergen Biosystems, Freemont, Calif.). Note that H50 is a selective surface and does not capture all protein in a sample, but a subset. The multi-well and array format can also be further expanded to higher density (384-well, 1536-well formats) microplates. In addition, the IgY compositions can also be used in microfluidics instruments, such as the LabChip 90 Electrophoresis System or the LabChip 3000 Drug Discovery System (Caliper Life Sciences, Hopkinton, Mass.).

### Example 14

### Mixing IgY Antibodies at Certain Ratio Before Covalently Linked to Solid Support

Individual IgY antibodies can be conjugated to solid supports to form individual IgY compositions. In addition, a group of different IgY antibodies can also be simultaneously linked to a solid support matrix. The groups of IgY antibodies can be mixed in certain ratios for optimized immunoaffinity separation of target proteins. One example is to conjugate the 12 IgY antibodies used in MIXED12 through a process that is different from that of Example 8. The 12 IgY antibodies against HSA, IgG, Fibrinogen, Transferrin, IgA, α2-Macroglobulin, IgM, α1-Antitrypsin, Haptoglobin, Apolipoprotein A-I, Apolipoprotein A-II, and α1-Acid Glycoprotein are first mixed in a ratio based on the relative abundance of these 12 proteins in serum/plasma and the capacity of individual IgY microbeads. The mixed population of antibodies is then oxidized with sodium meta-periodate (5 mg/ml) at room temperature for 30 minutes, followed by dialysis against Phosphate Buffered Saline (PBS) to remove residual oxidant. Oxidized IgY antibodies are incubated with UltraLink® Hydrazide beads (Pierce Product No. 53149) to obtain conjugation ratios of 10 to 15 mg IgY/ml beads. Conjugation is carried out at 4° C. overnight with rotation. After conjugation, the IgY-coupled microbeads are thoroughly washed with 1M NaCl, followed by Tris-Buffered Saline (TBS, 10 mM Tris-HCl, 0.15 M NaCl, pH 7.4), and stored as a 50% slurry in TBS with 0.01% NaN₃ at 4° C.

### Example 15

### Mixing IgY Antibodies at Certain Ratios for Best Binding Group Targets

One of the features of IgY microbead technology is to apply polyclonal IgY antibody mixture for capturing and separating groups of target proteins. In order to have the most effective binding between IgY microbeads and their corresponding protein or antigen targets, as well as the optimal capacity of the mixed IgY microbeads, it is preferred to define the two parameters for individual IgY microbeads.

One the is depletion (or reduction) efficiency of a target antigen which is dependent on the abundance of the target in human serum/plasma and avidity of the individual IgY against these targets. The other is the optimal coupling density of individual IgY on the microbeads, which is dependent on the size of target antigen.

For the top three abundant proteins in human serum/plasma, HSA, IgG and Transferrin, since the avidities of specific IgYs against these three targets based on the capacity assay (see the disclosure of capacity assay for individual microbeads in the specification) of individual IgY micromeads are high, therefore, more than 99% of depletion efficiency of these targets was set for the final mixed IgY microbeads.

To determine the optimal densities for individual IgY microbeads, different coupling densities for individual IgY microbeads were used ranging from 10 to 30 mg IgY/ml microbeads to find out which coupling density gives the highest capacity for individual IgY microbeads. For the optimal coupling densities of top three antigens in human serum, 15 mg Ig Y/ml microbeads coupling density was used for IgG antigen (MW: 150 kDa), while 20 and 25 mg IgY/ml microbeads coupling densities were used transferrin (MW: 75 kDa), HSA (MW 68 kDa). It was discovered that highest coupling density does not necessarily give the highest capacity, especially for large target antigens like IgG, IgA, IgM and 2α-Macroglobulin. Most likely, the interaction between IgYs on microbeads and large antigens in the sample solution could be easily saturated due to the steric hindrance of large size of the target antigen.

Once the capacity of individual IgY microbeads is determined with defined depletion efficiency and optimal coupling density, the different individual IgY microbeads were initial mixed in a certain ratio based on the relative amount of the targets in sample solution as well as the capacity of individual IgY microbeads for the targets. The capacity of mixed IgY microbeads was further tested in the same way as that used for the individual IgY microbeads. Fine adjustment of the ratio for the mixed IgY microbeads was conducted to have the highest capacity for the final products while achieving the desired depletion efficiency for the multiple targets.

Tables 6 and 7 provide the examples of the ratios of IgY antibodies to form products (e.g. Top2 and Top3) based upon plasma protein concentration.

TABLE 6
Example Ratio of Mixing IgY Microbeads against HAS and IgG

| Antigens | | | IgY Microbeads | IgY |
|---|---|---|---|---|
| (Target | % in the | Molecular | Volume (ml) as | Microbeads |
| Proteins) | Plasma | Weight (kDa) | packed bed | (ml) (%) |
| HSA | 54 | 68 | 8.0 | 80 |
| IgG | 17 | 150 | 2.0 | 20 |

TABLE 7
Example Ratio of Mixing IgY Microbeads against
HAS, IgG, and Transferrin

| Antigens | | | IgY Microbeads | IgY |
|---|---|---|---|---|
| (Target | % in the | Molecular | Volume (ml) as | Microbeads |
| Proteins) | Plasma | Weight (kDa) | packed bed | (ml) (%) |
| HSA | 54 | 68 | 3.5 | 70 |
| IgG | 17 | 150 | 1.0 | 20 |
| Transferrin | 3.3 | 75 | 0.5 | 10 |

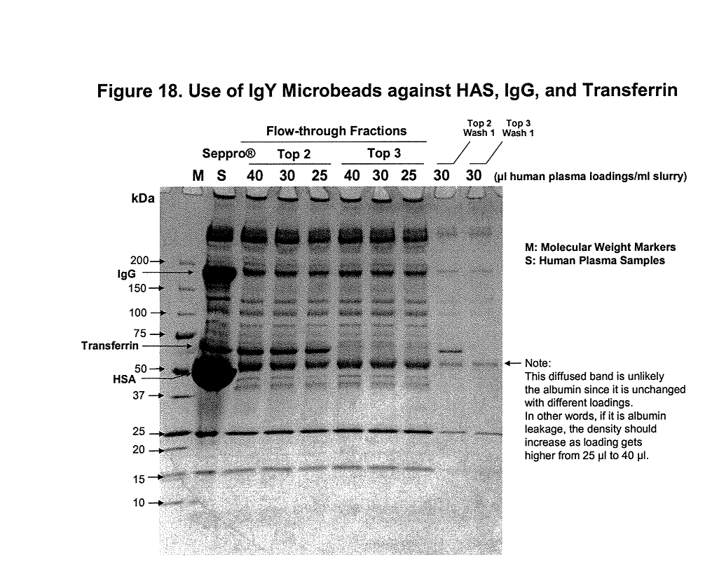

The mixed IgY microbeads against HSA and IgG (named Seppro® Top2) and those against HAS, IgG, and Transferrin (named Seppro® Top3) were tested in capturing and removing target proteins in plasma sample. Briefly, human plasma was diluted with dilution buffer (TBS: 10 mM Tris-HCl, 0.15 NaCl, pH 7.4) and added into spin columns containing either Seppro® Top2 or Seppro® Top 3, and incubated for 15 min at room temperature on a rotator followed by three washes with TBS. Bound proteins were eluted with 0.1 M Glycine-HCl, pH 2.5. Fractions were analyzed on 4-20% Gradient Tris-HCl SDD-PAGE (FIG. 18). The numbers, 25, 30, 40, indicate the three different loadings of neat human plasma μl per ml Seppro® slurry, respectively. It is clear that Seppro® Top2 effectively depleted HSA and IgG from human plasma in the flow-through fractions of lanes 3, 4, 5. Similarly, Seppro® Top 3 removed HSA, IgG, and Transferrin in the flow-through fractions of lanes 6, 7, 8. The positions of all three removed proteins in the gel were indicated by arrows. Note: one band near the position as HSA in all flow-through fractions is unlikely due to the leakage of HSA since the band kept unchanged with increased loadings. Lanes 9 and 10 were the first wash from Seppro® Top2 and Seppro® Top3 after depletions. M, Precision plus Protein Standards (Bio-Rad, Cat #161-0374). S, Normal Human Plasma form Innovative Research Inc. (Cat #IPLA-3).

All patents, patent applications, provisional applications, and publications referred to or cited herein are incorporated by reference in their entirety to the extent they are not inconsistent with the explicit teachings of this specification.

We claim:

1. An immunoaffinity separation composition for separating or reducing a first and second targets in a complex mixture, comprising:
a first immobilized affinity reagent and a second d immobilized affinity reagent,
wherein the first immobilized affinity reagent comprises a first affinity reagent being linked to a first solid support,
wherein the first affinity reagent is prepared using a first target and capable of binding to the first target,
wherein the second immobilized affinity reagent comprises a second affinity reagent being linked to a second solid support,
wherein the second affinity reagent is prepared using a second target and capable of binding to the second target, and
wherein a mixture ratio between the first immobilized affinity reagent and the second immobilized affinity reagent or between the first affinity reagent and the second affinity reagent is determined so as to efficiently reduce or separate the first and second targets.

2. The immunoaffinity separation composition of claim 1 wherein the first affinity reagent is orientedly linked to the first solid support and the second affinity reagent is orientedly linked to the second solid support.

3. The immunoaffinity separation composition of claim 1 wherein the affinity reagents are orientedly linked to the solid supports covalently or non-covalently.

4. The affinity separation composition of claim 1 wherein the first affinity reagent is a first IgY polyclonal antibody having a first Fc region and first Fab regions and the first target is a first antigen, and the second affinity reagent is a second IgY polyclonal antibody having a second Fc region and second Fab regions and the second target is a second antigen.

5. The affinity separation composition of claim 4 wherein the first IgY antibody is made by immunizing and boosting a first bird with the first antigen, covalently linked to the first solid support through the first Fc region, and capable of specifically binding to the first antigen through the first Fab regions, and the second IgY antibody is made by immunizing and boosting a second bird with the second antigen, is covalently linked to the second solid support through the second Fc region, and capable of binding to the second antigen through the second Fab regions.

6. The affinity separation composition of claim 5 wherein the first and second bird are chickens.

7. The affinity separation composition of claim 5 wherein the first and second solid supports contain hydrazide groups that form hydrazone bonds with the oxidized glycosylation moieties in the first and second Fc regions.

8. The affinity separation composition of claim 5 wherein the first and second antigens are protein, peptides, protein-protein complexes, protein-nucleotide complexes, protein-sugar/lipid complexes, biological complexes, nucleotides, cells, subcellular organelles, or microorganisms.

9. The affinity separation composition of claim 8 wherein the first antigen is a first protein and the second antigen is a second protein.

10. The affinity separation composition of claim 9 wherein the first and second proteins are human proteins.

11. The affinity separation composition of claim 10 wherein the first protein is selected from the group consisting of Albumin, IgG, Fibrinogen, Transferrin, IgA and IgM; the second protein is selected from the group consisting of Albumin, IgG, Fibrinogen, Transferrin, IgA and IgM; and the first protein is not the same as the second protein.

12. The affinity separation composition of claim 10 wherein the first protein is selected from the group consisting of Albumin, IgG, Fibrinogen, Transferrin, IgA, α2-Macroglobulin, IgM, α1-Antitrypsin, Haptoglobin, α1-Acid Glycoprotein, Apolipoprotein A-I and Apolipoprotein A-II, and High-Density Lipoprotein; the second protein is selected from the group consisting of Albumin, IgG, Fibrinogen, Transferrin, IgA, α2-Macroglobulin, IgM, α1-Antitrypsin, Haptoglobin, α1-Acid Glycoprotein, Apolipoprotein A-I and Apolipoprotein A-II, and High-Density Lipoprotein; and the first protein is not the same as the second protein.

13. The affinity separation composition of claim 1 wherein the first solid support is the same as the second support, the first affinity reagent is mixed with the second affinity reagent at the ratio first, and the mixed first affinity and second affinity reagents are then linked to the solid supports to form the first immobilized affinity reagent and the second immobilized affinity reagent.

14. The affinity separation composition of claim 1 wherein the first solid support is not the same as the second support, the first affinity reagent is linked with the first solid support to form the first immobilized affinity reagent, the second affinity reagent is linked with the second solid support to form the second immobilized affinity reagent, the first and second immobilized affinity reagents are then mixed at the ratio.

15. The affinity separation composition of claim 1 wherein the solid support has a surface material for linking the solid support to the affinity reagent, a defined shape and a core material for mediating separation of the targets from the complex mixture.

16. The affinity separation composition of claim 15 wherein the defined shape is a sphere or an area surface.

17. The affinity separation composition of claim 16 wherein said sphere is a microsphere or a nanosphere.

18. The affinity separation composition of claim 17 wherein said sphere is in multiplex format of a mixture of spheres.

19. The affinity separation composition of claim 16 wherein the area surface is a well or channel that can contain a solution or allow the solution to flow.

20. The affinity separation composition of claim 19 wherein the well a microplate well in a format of 96 wells, 384 wells, or 1536 wells on a plate.

21. The affinity separation composition of claim 15 wherein said surface material is a chemical or biological group that is capable of linking the solid support to the affinity reagent.

22. The affinity separation composition of claim 21 wherein the chemical group is a hydrazide.

23. The affinity separation composition of claim 21 wherein the biological group is the combination of biotin and avidin, or biotin and streptavidin.

24. The affinity separation composition of claim 15 wherein said core material is an acrylamide/azlactone copolymer, a polystyrenedivinylbenzene, a polystyrene, an agarose, a polymer, a resin, a polyester, a metal, a paramagnetic material, a magnetic material or mixtures thereof.

25. The affinity separation composition of claim 24 wherein the core material is coated with the surface material and in the defined shapes which is a sphere or an area surface.

26. The affinity separation composition of claim 15 wherein the first and second solid supports are placed in a device to mediate separation of targets from the complex mixture.

27. The affinity separation composition of claim 26 wherein the device is a chromatographic column, a multiple-well plate, or a microfluidic apparatus.

28. The affinity separation composition of claim 27 wherein the column is a spin column, a conventional liquid chromatographical column, an FPLC or HPLC column, a tip or a combination of them operable through a manual or automated process.

29. The affinity separation composition of claim 27 wherein the multiple well plate is a plate or micro-plate containing 8-wells, 16-wells, 64-wells, 96-wells, 384-wells, or 1536-wells per plate.

30. The affinity separation composition of claim 1 wherein the complex mixture is plasma, serum, body fluid derived from tissue, cerebrospinal fluid, bronchial alveolar lavage, vitreous humor, nipple aspirate, or urine.

31. A method of fractioning, separating, depleting a plurality of targets or enriching a plurality of non-targets in a complex mixture, comprising the steps of:
a) contacting the complex mixture with the affinity separation composition of claim 1;
b) collecting a flow-through fraction, wherein the concentration of the first and second targets in the flow-through fraction has been reduced compared to the complex mixture.

32. The method of claim 31 further comprising a step of collecting an elution fraction by recovering the first and second targets which have bound to the immobilized affinity reagents after the complex mixture is contacted with the affinity separation composition.

33. A method of identifying an association between proteins in a biological sample which comprises:
a) contacting the biological sample with the affinity separation composition of claim 9;
b) collecting an elution fraction which contains the first and second proteins; and
c) analyzing the elution fraction to determine if at least one other protein in the biological sample is associated with the first or the second protein.
